(19) United States
(12) Patent Application Publication
(10) Pub. No.: US 2021/0147855 A1
(43) Pub. Date: May 20, 2021

(54) OPTOGENETIC CIRCUITS FOR CONTROLLING CHEMICAL AND PROTEIN PRODUCTION IN ESCHERICHIA COLI

(71) Applicant: THE TRUSTEES OF PRINCETON UNIVERSITY, Princeton, NJ (US)

(72) Inventors: José L. Avalos, Princeton, NJ (US); Makoto A. Lalwani, San Jose, CA (US); César Carrasco-López, Lawrence Township, NJ (US)

(73) Assignee: THE TRUSTEES OF PRINCETON UNIVERSITY, Princeton, NJ (US)

(21) Appl. No.: 17/097,484

(22) Filed: Nov. 13, 2020

Related U.S. Application Data
(60) Provisional application No. 62/935,267, filed on Nov. 14, 2019.

(51) Int. Cl. C12N 15/63; C12N 15/85

(52) U.S. Cl. CPC C12N 15/635; C12N 2830/005; C12N 15/85

(57) ABSTRACT

Disclosed herein are optogenetic circuits for the bacterium Escherichia coli that induce gene expression in darkness and repress it under blue light. Applying them to metabolic engineering improves chemical production compared to chemically induced controls in light-controlled fermentations. More particularly, these circuits can be used to control protein production with light. The system and method use light as a suitable alternative to chemical induction for microbial production of chemicals and proteins.

### CROSS-REFERENCE TO RELATED APPLICATIONS

This application claims priority to U.S. Provisional Patent Application No. 62/935,267, filed Nov. 14, 2019, which is hereby incorporated in its entirety by reference.

### STATEMENT REGARDING FEDERALLY SPONSORED RESEARCH OR DEVELOPMENT

This invention was made with government support under Grant No. CBET-1751840 awarded by the National Science Foundation and Grant No. DE-SC0019363 awarded by the Department of Energy. The government has certain rights in the invention.

### SEQUENCE LISTING

A Sequence Listing filed electronically herewith is also hereby incorporated by reference in its entirety (File Name: PRIN-71502_ST25.txt; Date Created: Nov. 12, 2020; File Size: 19,446 bytes.)

### BACKGROUND

The long history of Escherichia coli as a model organism makes it a preferred host for many biotechnological applications, including metabolic engineering and recombinant protein production. The abundance of genetic tools and wealth of knowledge about its physiology, genetics and metabolism have made E. coli an organism of choice across the spectrum of metabolic engineering, from proofs of principle to large-scale industrial processes. However, metabolic engineering in E. coli still faces important challenges that may be addressed with new technological improvements.

A key hurdle arises when engineered pathways cause cellular toxicity, metabolic burden or competition with cell growth, resulting in poor productivity. This can be addressed by dynamically controlling fermentations through a growth phase (in which engineered pathways are repressed to focus the cellular metabolism on biomass build-up) and a production phase (in which engineered pathways are induced for product biosynthesis). However, pathway productivities can be greatly impacted by the timing, rates and levels of induction, which can be difficult to control with chemical inducers.

The most commonly used inducible system in E. coli is based on the lac operon. In the absence of lactose, the LacI repressor binds to lac operator (lacO) sites upstream of genes involved in lactose metabolism, repressing them. Allolactose binding to LacI causes it to dissociate from lacO sites, thus allowing gene transcription. For decades, this system has been exploited for dynamic control of gene expression in E. coli, using lacO sites to recruit LacI to a variety of promoters, and isopropyl β-d-1-thiogalactopyranoside (IPTG), a non-metabolizable allolactose mimetic, as an inducing agent. These features of the lac operon provide an opportunity to develop optogenetic circuits to control gene expression in E. coli with light.

Several optogenetic systems have been developed in E. coli. Among these, the photoreceptor signaling cascade encoded in the pDawn plasmid seemed particularly adept at harnessing the lac operon. The pDawn system is derived from an engineered blue light-responsive photosensory histidine kinase YF 1 and its cognate response regulator FixJ from Bradyrhizobium japonicum. In the dark, YF 1 phosphorylates FixJ, which then activates the P_{FixK2} promoter to express the λ phage repressor cI. This repressor in turn prevents transcription from its cognate P_{R} promoter, which controls genes of interest. Conversely, blue light (˜470 nm) induces YF1 phosphatase activity, thus reversing FixJ phosphorylation and cI expression. With this design, genes controlled by P_{R} are repressed in the dark by cI and expressed in blue light by the absence of this repressor.

Improvements in these circuits are therefore desirable and useful.

### BRIEF SUMMARY

The presently disclosed invention is drawn to a system and method for optogenetic control of the lac operon using a series of circuits called OptoLAC, in which lacI is controlled by, e.g., the P_{R} promoter of the pDawn system. These optogenetic circuits can optionally bestow light controls on promoters originally designed to be IPTG-inducible.

A first aspect of the present disclosure is drawn to a system containing (i) a first sequence encoding a first repressor, under a first promoter, the first promoter being light-controllable promoter; a second sequence encoding a second repressor, under a second promoter, the second promoter being controllable by the first repressor; and a third sequence encoding a gene of interest under a third promoter, the third promoter being controllable by the second repressor. Optionally, the first promoter is controlled by a two-component system (such as a system comprising the transcription factor FixJ, which in turn is controlled by the light responsive kinase/phosphatase YF1) or a one-component system (the LOV-domain-containing EL222 transcription factor fused to an activation domain (such as VP16).

Optionally, the first repressor is phage repressor cI, and/or the second repressor is a Lad repressor. Optionally, the third promoter is a lacO-operator-containing promoter. Optionally, the first repressor and/or the second repressor is fused to a sequence encoding for a degradation tag.

Optionally, the first promoter is also controllable by the second repressor (that is, the first promoter is also controllable by the second repressor, such that when the second repressor is expressed, it not only represses the expression of the gene of interest, it also represses the expression of first repressor). Optionally, the first promoter comprises P_{FixK2}.

Optionally, the gene of interest is one or more genes involved in the biosynthesis of a chemical compound, such as mevalonate or isobutanol. Optionally, the gene of interest is one or more genes involved in production of a recombinant protein, and may comprise a fluorescent protein, FdeR, TmSir2, Pdc1p, nanobodies, monobodies, LlilvD, HRAS, or a combination thereof

Optionally, the system is present in at least one plasmid, each plasmid being free of endogenous and constitutive sequences encoding the first or second repressors. When present in more than one plasmid, each plasmid includes at least one of sequence selected from the first sequence, second sequence, and third sequence.

Optionally, the system is present in or integrated into the genome of an engineered microorganism, such as E. coli, the engineered microorganism being free of endogenous and constitutive sequences encoding the first or second repressors.

Optionally, the system is split into an organism and one or more plasmids. For example, optionally a portion of the system is integrated into the genome of an engineered microorganism (which is free of endogenous and constitutive sequences encoding the first or second repressors). This could be, e.g., an organism containing the first and second sequences. The remaining portion of the system is present in at least one plasmid, where each plasmid includes at least one of the three sequences as described previously. The plasmid would preferably later be used to transform the microorganism.

Also disclosed is a method for controlling chemical or protein production. The method first requires growing engineered microorganism cells (that are free of endogenous and constitutive sequences encoding the first or second repressors) containing the system described previously. The growth occurs under a first lighting condition, typically where the third promoter is repressed. The gene of interest is expressed by adjusting the first lighting condition and causing the first repressor to be expressed, which prevents the second repressor from being expressed, which allows the third promoter to be activated.

Optionally, the lighting condition is adjusted when the optical density at a wavelength of 600 nm (OD₆₀₀) of the microorganism is determined to be within a predetermined range, such as between 0.1 and 2.

Optionally, the method also includes obtaining cell-free supernatant after the gene of interest has been expressed, the supernatant containing a chemical of interest expressed by the gene of interest, and then optionally analyzing a sample of the cell-free supernatant. Optionally, the method also includes collecting the cells and lysing the cells to obtain a chemical or protein of interest produced by the gene of interest.

Optionally, the method involves repeatedly turning on the light source for a first period of time T₁, then turning off the light source for a second period of time T₂, where optionally T₁/(T₁+T₂) is between about 0.001 and about 0.1.

Also disclosed is a kit, containing at least one engineered microorganism and one or more plasmids. The engineered microorganism has the first and second sequences integrated into its genome, while the plasmids contain a promoter controlled by the second repressor, sites to integrate one or more genes of interest, and optionally at least one sequence to fuse tags (e.g., for affinity purification, overexpression, secretion, detection, etc.). The plasmids are free of constitutive and endogenous sequences encoding the first or second repressors.

### BRIEF DESCRIPTION OF DRAWINGS

FIG. 1 is a depiction of a prior art pDawn-based system.

FIG. 2A-2B are depictions of a disclosed system, in the dark (2A) and in the light (2B).

FIG. 2C is a depiction of a disclosed system that contains a feedback loop.

FIG. 3 is a flowchart of a disclosed method

FIG. 4A is a graph showing GFP expression from P_{BBA} (EMAL231) or P_{T5-lacO} controlled by OptoLAC1 (EMAL68), OptoLAC2 (EMAL69) or OptoLAC3 (EMAL230) in blue light (white) or darkness (black). The fold of induction of each OptoLAC circuit is shown.

FIGS. 4B-4D are graphs depicting the effectiveness of the disclosed optogenetic circuits, based on the production of GFP under the control of the circuit. FIG. 4B shows GFP expression under a constitutive P_{BBA} promoter (EMAL231) or where lacI expression is controlled by pDawn (EMAL57). FIG. 4C shows GFP expression under a constitutive P_{BBA} promoter (EMAL231), or from PTF-lacO strains containing the pDawn system controlling LacI fused to SsrA tags terminating in LAA (EMAL68), AAV (EMAL69), or ASV (EMAL71); the folds of induction are shown. FIG. 4D shows GFP expression by the P_{Fix2} or P_{FixK2_lacO} promoters using pDusk (EMAL152 and EMAL153, respectively.

FIG. 5 is a graph of integrated peak volumes of LacI protein levels quantified via Western blot for various embodiments of OptoLAC circuits (OptoLAC1, OptoLAC2, and OptoLAC3).

FIG. 6 is a graph showing GFP expression from P_{T5-lacO} controlled by OptoLAC1 (white, EMAL68), OptoLAC2 (black, EMAL69), or OptoLAC3 (striped, EMAL230) under blue light with different concentrations of IPTG added at the time of inoculation.

FIG. 7 is a graph showing GFP expression at 18° C. from P_{BBA} (EMAL231) or P_{T5-lacO} controlled by OptoLAC1 (EMAL68), OptoLAC2 (EMAL69), or OptoLAC3 (EMAL230) under blue light or darkness.

FIG. 8A is a graph showing GFP expression from PBBA (EMAL231) or from P_{T5-lacO} controlled by OptoLAC1 (EMAL68) or OptoLAC3 (EMAL230), under full light, 100 s on/1,000 s, 10 s on/1,000 s and full darkness.

FIG. 8B is a graph showing GFP expression from PBBA (EMAL231) or controlled by OptoLAC1 from P_{lacUV5} (EMAL267) or P_{trc} (EMAL268).

FIG. 8C is a graph showing GFP expression from P_{T5-lacO} controlled by OptoLAC1 (EMAL68) in M9 medium+2% glucose, M9+2% xylose, LB medium and SOB medium.

FIG. 8D is a graph of the time course of GFP expression from P_{T5-lacO} controlled by OptoLAC1 (EMAL68; long dashed line), OptoLAC2 (EMAL69; short dashed line), OptoLAC3 (EMAL230; dash-and-double dot line) or IPTG (EMAL77; dotted line) compared to a negative control lacking GFP (EMAL229; solid black line). P=0.000154.

FIG. 8E is a graph of GFP expression from systems shown in FIG. 8D at 12 h, including uninduced controls where cultures were kept in blue light or without IPTG. From left to right: P<0.00001, P<0.00001,P<0.00001,P<0.00001.

FIG. 8F is a graph of the time course of destabilized GFP expression from P_{T5-lacO} controlled by OptoLAC1 (EMAL343) in blue light (dotted line) or darkness (solid line), from cells previously grown in dark conditions.

FIG. 9 is a photo showing spatial control of GFP expression on an LB agar plate, containing a lawn of EMAL68 (OptoLAC1 driving GFP) illuminated with a projection of a tiger image. Scale bar is 1 cm.

FIG. 10A is a graph of isobutanol production controlled with OptoLAC1 (EMAL199) when switching cultures from blue light to darkness at different cell densities (ρₛ) or uninduced (“Light”) or induced with IPTG (EMAL201) at optimal cell density.

FIG. 10B is a graph of isobutanol production controlled with OptoLAC3 (EMAL239) when switching cultures from blue light to darkness at different cell densities (ρₛ) or uninduced (“Light”) or induced with IPTG (EMAL201) at optimal cell density.

FIG. 11 is a graph of mevalonate production controlled with OptoLAC1 (EMAL208) when switching cultures from blue light to darkness at different cell densities (ρs) or uninduced (“Light”, as well as induced with IPTG (EMAL135).

FIG. 11B is a graph of mevalonate production controlled with OptoLAC2 (EMAL209) when switching cultures from blue light to darkness at different cell densities (ρs) or uninduced (“Light”, as well as induced with IPTG (EMAL135).

FIG. 11C is a graph of light-controlled mevalonate production and optical densities (OD₆₀₀) in a 2-l bioreactor.

FIG. 12A shows gel blots for a time course of light-controlled YFP production in E. coli B strain (OptoBL), induced by switching cultures from blue light to darkness using OptoLAC1B (EMAL284, top) or adding IPTG (EMAL283, bottom).

FIG. 12B shows gel blots for a time course of light-controlled FdeR production, induced by switching cultures from blue light to darkness using OptoLAC2B (EMAL336, top) or adding IPTG (EMAL329, bottom).

FIGS. 12C-12E show the tunability of light-controlled FdeR production using different doses of light or concentrations of IPTG, resolved and quantified via western blot.

FIG. 13A-13F show a compilation of gels illustrating recombinant protein production using OptoLAC circuits. Cultures were grown under continuous blue light until exponential phase, then moved to darkness (wrapped in aluminum foil) and grown for 8 hours at 37° C. (13A-13C) or 20 hours at 30° C. (13D-13F). FIGS. 13A and 13D show gels for TmSir2=sirtuin from Thermotoga maritima; F6P1=Pdc1p-binding nanobody; 3K2M Mono=SH2 domain-binding monobody; and YFP_3K2M Binder=YFP-SH2 fusion. FIGS. 13B and 13C show gels for LlilvD=dihydroxy-acid dehydratase from Lactococcus lactis; HRAS=GTPase HRas; Pdc1p=pyruvate decarboxylase from Saccharomyces cerevisiae. Controls (13C, 13F) for each of the proteins being produced were grown in ambient light and induced in exponential phase by the addition of IPTG to a final concentration of 1 mM.

Unless otherwise noted, for each graph, * P<0.05, ** P<0.01, *** P<0.0001.

### DETAILED DESCRIPTION

Disclosed is a system and method for optogenetic control of an gene of interest via a series of circuits. These optogenetic circuits can optionally bestow light controls on promoters originally designed to be IPTG-inducible, and that these circuits can replace IPTG with light to control engineered metabolic pathways for chemical and/or protein production.

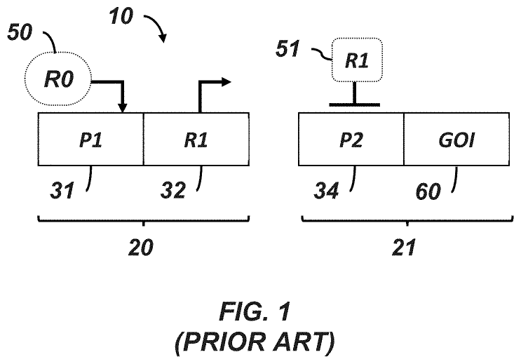

Traditional pDawn systems can be described with respect to FIG. 1. There, the prior art system 10 contains two nucleotide sequences 20, 21. The first nucleotide sequence 20 contains a sequence 32 encoding for a first repressor 51 (cI) under a first promoter 31 (P_{FixK2}). The first promoter 31 is indirectly controlled by light; as described previously, in the dark, YF1 phosphorylates FixJ (50) which thus activates the first promoter, while in the light the FixJ is dephosphorylated by YF1 and therefore does not activate it. The second nucleotide sequence contains a sequence that contains a gene of interest (GOI) 60 under a second promoter 25 (P_{R}) which is repressed in the presence of cI. As described previously, in the dark, the system does not express the GOI, and in the light, the GOI is expressed.

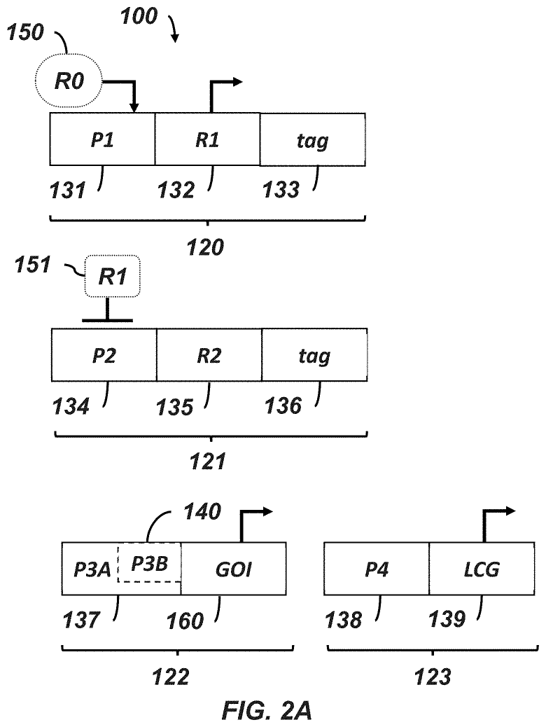

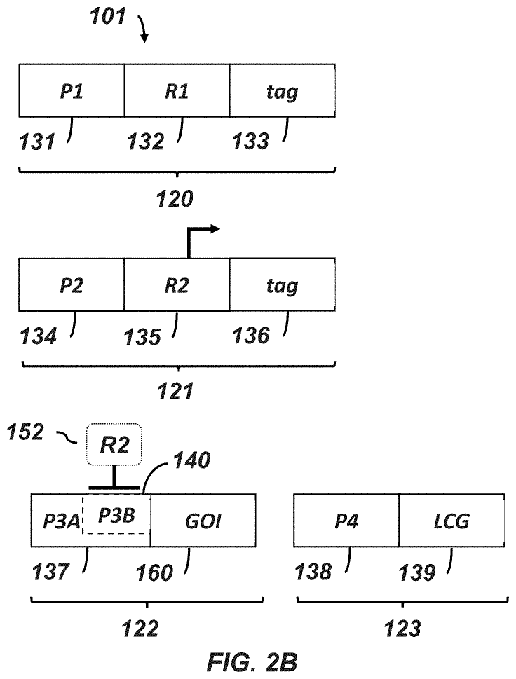

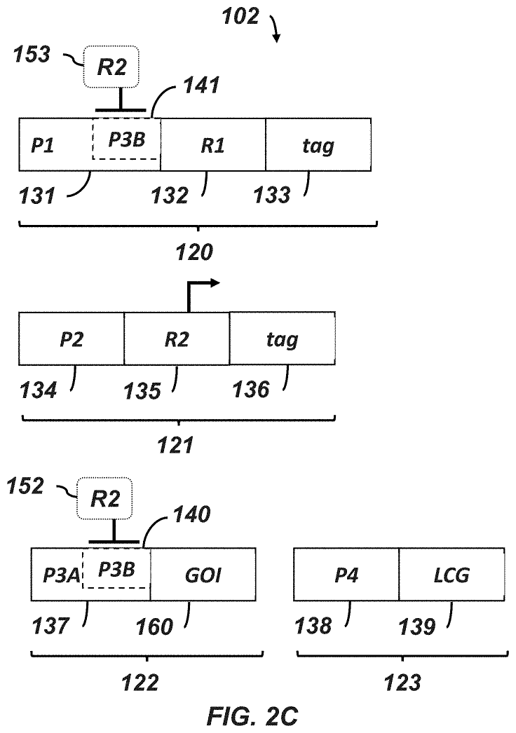

A first aspect of the present disclosure is drawn to the optogenetic circuits, which can be described with respect to FIGS. 2A and 2B, and generally requires at least three nucleotide sequences.

In FIG. 2A, a first nucleotide sequence 120 encodes a first repressor 132 (such as phage repressor protein cI), under a first promoter 131 that is light-controllable. Optionally, the first nucleotide sequence may encode a degradation tag 133 fused to the first repressor 132.

The nucleotide sequence encoding the first repressors 132 are known to those of skill in the art. A preferred embodiment comprises a sequence that encodes for cI, although any repressor that can repress a promoter is envisioned.

For degradation tags 133, any appropriate degradation tag known to a skilled artisan can be used. As known in the art, degradation tags are short peptide sequences that mark a protein for degradation by the cell. The degradation tag decreases the protein half-life (the interval time it takes for the level of the protein to decay to half its initial value).

The first promoter 131 can be any promoter that allows the first sequence to express the first repressor (and the optional degradation tag) when either (a) the system is exposed to one or more wavelengths of light (such as blue light) or (b) the system is not exposed to such wavelengths.

The first promoter may be controlled by light through a two-component system. That is, in some embodiments, the light (or lack thereof) causes a light-responsive protein to modify a transcription factor that controls the first promoter. A preferred embodiment of a two-component system light-control is a light-responsive YF1 protein which phosphorylates FixJ in the dark and dephosphorylates it in blue light. Thus, in the dark, phosphorylated FixJ can activate the P_{FixK2} promoter from the pDawn system, but not in the light, when FixJ is dephosphorylated. There, the promoter relies on blue light causing YF1 to phosphorylate FixJ, which can then activate P_{FixK2.}

The first promoter may be directly controlled by a one-component system. That is, in some embodiments, the light (or lack thereof) causes a morphology change directly on the transcriptional factor that controls the first promoter. For example, in a system that is capable of expressing EL222 fused to an activation domain, such as VP16, and a C120 promoter can be used. When exposed to blue light, VP16-EL222 uncages the HTH-DBD (Helix-Turn-Helix DNA-Binding Domain), facilitating homodimerization of two VP16-EL222 proteins which then bind to the C120 promoter to activate them. Other known optogenetic techniques, including the use of LOV domains and Cryptochromes, is also envisioned.

In some embodiments, the first promoter is one that can be activated when the system it is a part of is exposed to one or more wavelengths of light, and repressed or not otherwise activated (or activated) when not exposed. In other embodiments, the first promoter is controlled in the reverse fashion—repressed or not activated when exposed to one or more wavelengths of light, and activated when not exposed.

The system also include a second nucleotide sequence 121 encoding a second repressor 135 (such as a LacI repressor) under a second promoter 134, where the second promoter is controllable by the first repressor. Optionally, the second nucleotide sequence may encode a degradation tag 136 fused to the second repressor 135.

The nucleotide sequence encoding the second repressors 135 are known to those of skill in the art. A preferred embodiment comprises a sequence that encodes for the LacI repressor, although any repressor that is different from the first repressor, and can bind to a promoter, is envisioned.

For degradation tag 136, any appropriate degradation tag known to a skilled artisan can be used. In some embodiments, degradation tag 136 is the same as degradation tag 133. In other embodiments, degradation tag 136 is different from degradation tag 133.

The second promoter 134 can be any promoter known to skilled artisans that allow the promoter to be repressed in the presence of the first repressor. In preferred embodiments, the promoter is repressed in the presence of cI. In more preferred embodiments, the cI-controlled promoter 134 is the λ major lytic promoter, pR, found in, e.g., pDawn.

The system also includes a third nucleotide sequence 122 containing a gene of interest (GOI) 160 under a third promoter 137 (P3A).

The third promoter should comprise a sequence 140 (P3B) that is controllable by the second repressor, typically downstream. The sequence controllable by the second repressor may be, e.g., a lacO operator-containing promoter. For example, the lacO-containing promoter could be, e.g., P_{T5-lacO}P_{lacUV5}, P_{trc}, P_{L_lacO1} and P_{T7}.

No substantive restrictions exist on gene of interest. The gene of interest may be, e.g., one or more genes involved in the biosynthesis of a chemical compound. In some embodiments, the gene of interest is one or more genes involved in the biosynthesis of, e.g., C₃-C₂₀ ketones such as acetone, butanone, 2-undecanone, and 2-tridecanone; alcohols such as ethanol, isopropanol, isobutanol, 1-butanol, 2-methyl-1-butanol, 3-methyl-1-butanol, and 2-phenylethanol; hydroxy fatty acids such as mevalonate, 9-hydroxydecanoic acid, 10-hydroxyhexadecanoic acid, 11-hydroxydodecanoic acid, or 12-hydroxyoctadecanoic acid; lactates; succinates; and/or acetates.

The gene of interest may also be, e.g., one or more genes involved in production of a recombinant protein, which may comprise, e.g., a fluorescent protein, an enzyme, a transcription factor optionally fused to a cleavage tag, affinity tag, and/or solubility enhancing tag. For example, a pharmaceutically useful proteins fused to an affinity tag may be expressed from a gene of interest, after which the proteins could be purified using techniques known to those of skill in the art.

The recombinant protein may comprise, e.g., FdeR, TmSir2, Pdc1p, nanobodies, monobodies, LlilvD, and/or HRAS.

The system may optionally contain a fourth nucleotide sequence 123, which may include one or more light control-related genes (LCG) 139 that express one or more compounds that are utilized for light control of the first promoter. The LCGs are under a fourth promoter 138. The LCGs may be, e.g., YF1-FixJ, or EL222, to allow the system to express components that would inherently need to be present, based on the choice of first promoter.

In some embodiments, the system is present in one or more plasmids. Each plasmid should be free of endogenous and constitutive sequences encoding the first or second repressor. The system may be broken up into 2 or more plasmids, where each plasmid independently contains one or more of the above-described nucleotide sequences. For example, in one embodiment, a first plasmid contains the first nucleotide sequence, a second plasmid contains the second nucleotide sequence, a third plasmid contains the third nucleotide sequence, and a fourth plasmid contains the optional fourth nucleotide sequence.

In some embodiments, the system is present in an engineered microorganism. The engineered microorganism should be free of endogenous and constitutive sequences encoding the first or second repressor.

Any appropriate microorganism may be utilized.

In some embodiments, the engineered microorganism is a bacterium. In some embodiments, the microorganism is from a species of Escherichia, such as E. albertii, E. coli, E. fergusonii, E. hermannii, or E. vulneris, and is preferably E. coli. In some embodiments, the microorganism is from a species of Bacillus, such as B. alcalophilus, B. cereus, B. halodurans, B. siamensis, B. subtilis, or B. thuringiensis, and is preferably B. subtilis. In some embodiments, the microorganism is from a species of Streptomyces, such as S. antibioticus, S. aureofaciens, S. avermitilis, S. chartreusis, S. coelicolor, S. lysosuperificus, S. rimosus, S. scabies, S. torulosus, or S. venezuelae. In some embodiments, the microorganism is from a species of Pseudomonas, such as P. aeruginosa, P. chlororaphis, P. fluorescens, P. pertucinogena, P. putida, P. stutzeri, P. syringae, or P. teessidea. In some embodiments, the microorganism is from a species of Corynebacterium, such as C. amycolatum, C. bovis, C. diphtheriae, C. efficiens, C. glutamicum, C. jeikeium, C. macginleyi, C. matruchotii, C. minutissimum, C. pseudotuberculosis, C. renale, C. striatum, C. ulcerans, C. urealyticum, C. uropygiale, and C. xerosis.

In some embodiments, the engineered microorganism is a yeast. In some embodiments, the microorganism is from a species of Candida, such as C. albicans, C. glabrata, C. guilhermondii, C. krusei, C. lusitaniae, C. parapsilosis, C. rugosa, or C. tropicalis. In some embodiments, the microorganism is from a species of Aspergillus, such as A. fumigatus or A. nidulans. In some embodiments, the microorganism is from a species of Cryptococcus, such as C. neoformans. In some embodiments, the microorganism is from a species of Saccharomyces, such as S. cerevisiae.

In some embodiments, the engineered microorganism is a fungi. In some embodiments, the microorganism is from a species of Penicillium, such as P. jensenii or P. nalgiovense.

Existing IPTG-inducible vectors using, e.g., one or more different lacO-containing promoters (such as P_{T5-lacO}, P_{lacUV5}, P_{trc}, P_{L_lacO1} and P_{T7}) could be adapted to work with OptoLAC circuits and ΔlacI strains (e.g., OptoMG or OptoBL) for optogenetic control. This only involves removing any had copy and ensuring that each plasmid uses a unique selection marker and origin of replication. One of skill in the art will recognize that other third promotor/second repressor combinations could be used, provided an appropriate deletion strain was provided.

In some embodiments, the system may include both at least one engineered microorganisms and one or more plasmids. A portion of the system is integrated into the genome of the microorganism (which is free of endogenous and constitutive sequences encoding the first or second repressors). The remaining portion of the system is present in at least one plasmid (each plasmid being free of endogenous and constitutive sequences encoding the first or second repressors). That is, each plasmid includes the first sequence, second sequence, and/or third sequence (and optionally, the fourth sequence) as described above.

For example, in an intermediate stage, a skilled artisan may have the first and second sequences integrated into the genome of a microorganism, and have a plasmid they have modified to include a third sequence, which can then be used to transform the microorganism.

Thus, another aspect of the present disclosure is for a kit containing one or more strains of at least one engineered microorganism, and one or more plasmids. Each strain of a microorganism will have a first nucleotide sequence integrated that encodes a first repressor under a first light-controllable promoter, and a second nucleotide sequence encoding a second repressor under a second promoter, the second promoter being controllable by the first repressor. Each plasmid would contain a different promoter controlled by the second repressor, and optionally have different multicloning sites for integrating one or more genes of interest, optionally at least one sequence to fuse tags, and a different origin of replication. Each plasmid would be free of endogenous and constitutive sequences encoding the first or second repressors.

These systems operate in a particular manner. One specific example can be described in reference to FIGS. 2A and 2B. These systems contain the above-described three sequences (120, 121, 122), as well as a fourth sequence (123) that expresses YF1-FixJ. In the dark, YF1 phosphorylates FixJ. The phosphorylated FixJ 150 can activate the first promoter 131 (in this example, P_{FixK2}), causing the first repressor (in this example, cI 151) to be expressed. The cI 151 then represses the second promoter 134 (here, P_{R}), preventing the second repressor 135 from being expressed. Without the second repressor being expressed, the third promoter 137, 140 (in this example, lacO operator-containing promoter P_{T5-lacO}) is activated, allowing the gene of interest 160 to be expressed.

Conversely, when exposed to blue light, FIG. 2B shows how the expression of the gene of interest is repressed. There, exposure to blue light causes YF1 to dephosphorylate FixJ, which is thus unable to activate P_{FixK2} (and therefore P_{R} is not repressed), so lacI 152 is expressed, which represses the lacO operator-controlled promoter, and the gene of interest is not expressed.

These disclosed systems may have some degree of “leakiness”. For example, because of the underlying biological nature of the phosphorylating and transcriptional process, some FixJ might activate P_{FixK2} even when exposed to blue light, causing partial transcriptional repression of lacI. As such, there may be a need to reduce the “leakiness” of the disclosed optogenetic circuits.

An alternate approach can be seen in reference to FIG. 2C, that contains a feedback loop for reducing leakiness. There, the system is the same as in FIGS. 2A and 2B, except that the first promoter 131, 141 in the first nucleotide sequence 120 now contains a sequence 141 (P3B) controlled by the second repressor (such as a lacO operator sequence) that is downstream of the original first promoter 131 (P1, such as P_{FixK2}). This sequence 141 is typically identical to the sequence 140 (P3B) in the third nucleotide sequence 122—that is if one lacO operator sequence is used in the third nucleotide sequence 122, the same lacO operator sequence would be used in the first nucleotide sequence 120 for the feedback loop. This sequence allows for additional control of the expression of the first repressor (such as cI). As can be seen, when the second repressor (such as LacI repressor) is expressed, it will not only repress the lacO operator-containing promoter 137, 140 (here, P_{T5-lacO}) in the third nucleotide sequence 122, it will also repress the expression of the first repressor. Essentially, in this example of a feedback loop, first promoter 131 was converted to a lacO operator-containing light-controllable promoter 131, 141. In this way, when exposed to blue light, the production of the second repressor acts as a feedback control to reduce the expression of the first repressor further increasing expression of the second repressor even if, e.g., some FixJ is still active in the light.

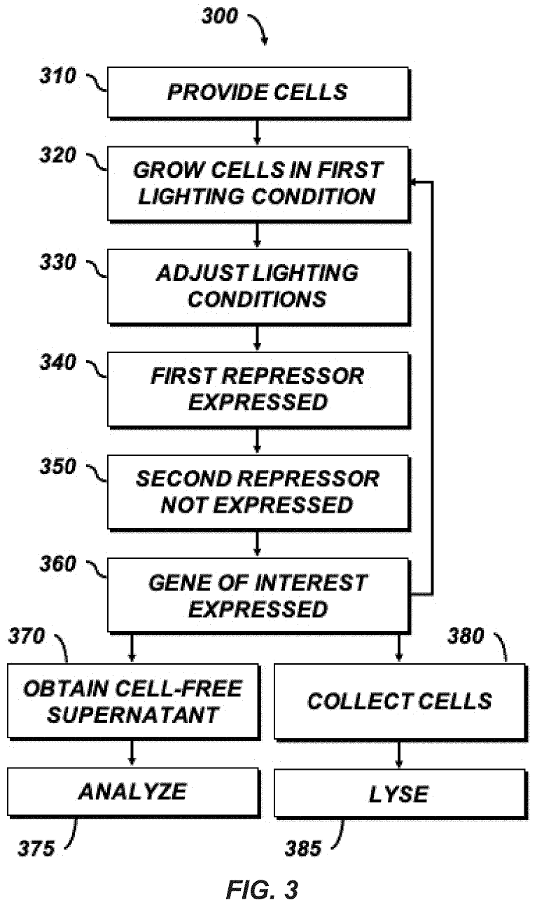

A second aspect of the present disclosure is a method for controlling chemical or protein production. This can be seen in reference to FIG. 3. There, the method 300 involves providing cells of at least one engineered microorganism 310 containing the disclosed system of three (and optionally four) nucleotide sequences as described above. As discussed previously, the engineered microorganism should be free of endogenous and constitutive sequences encoding the first or second repressors.

With those cells, a skilled artisan can grow 320 the cells under a first lighting condition. Under this first lighting condition, the second repressor (e.g., LacI) is expressed, which prevents the gene of interest from being expressed.

At some point thereafter, the skilled artisan can then alter the lighting conditions 330. This will cause expression of the first repressor 340, which represses the second promoter, preventing the expression of the second repressor 350. Without the second repressor, the third promoter is not repressed, which allows the gene of interest to be expressed 360.

In some embodiments, the lighting conditions are adjusted when the optical density at a wavelength of 600 nm (OD600) of the microorganism is determined to be within a predetermined range, such as between 0.1 and 2. In some embodiments, the lighting conditions are adjusted when the OD600 is ≥0.1, ≥0.2, ≥0.3, ≥0.4, ≥0.5, or ≥0.6, and ≤3, ≤2.5, ≤2, ≤1.5, ≤1.25, or ≤1, and any combination thereof.

In some embodiments, such as when the gene of interest is involved in the biosynthesis of a chemical compound, the method may further comprise obtaining cell-free supernatant 370, and then analyzing a sample of the cell-free supernatant 375. Methods for obtaining cell-free supernatant are known in the art.

In some embodiments, such as when the gene of interest is involved in the production of a protein (e.g., a recombinant protein), the method may further comprise collecting the cells 380 and lysing the cells 385, to measure protein production and/or purify the protein(s).

In some embodiments, the system is controlled by repeatedly adjusting the lighting conditions to fine-tune expression of the gene of interest. This can be done, by, e.g., turning on a light source for a first period of time T1, then turning off the light source for a second period of time T2. Preferably, the time period for expressing the gene of interest is longer than the time period for non-expression. For example, in some preferred embodiments, T1/(T1+T2) is between about 0.001 and about 0.1.

### EXAMPLES

Optogenetic circuits to control the lac operon. To develop light controls for E. coli, lacI was placed under the cI-controlled PR promoter of pDawn, such that genes of interest are repressed in the light and induced in the dark. To ensure control over total LacI levels, a ΔlacI E. coli K-12 MG1655 strain (EMAL52, hereafter OptoMG) was transformed with plasmids to control lacI expression with pDawn (pMAL288), and superfolder green fluorescent protein (GFP) expression with the IPTG-inducible promoter PT5-lacO (pMAL292), resulting in strain EMAL57.

Plasmids were cloned into E. coli strain DH5a made chemically competent using the Inoue method49. Transformants were inoculated on LB agar plates at 37° C. with appropriate antibiotics: 100 μg ml-1 ampicillin, 100 μg ml-1 carbenicillin, 50 μg ml-1 kanamycin, 34 μg ml-1 chloramphenicol or 50 μg ml-1 spectinomycin. Epoch Life Science Miniprep, Omega Gel Extraction and Omega PCR purification kits were used to extract and purify plasmids and DNA fragments. Backbones and inserts were either digested using restriction enzymes purchased from NEB or PCR-amplified using Q5 polymerase from NEB or CloneAmp HiFi PCR premix from Takara Bio. One-step Gibson isothermal assembly reactions were performed based on previously described protocols. Primers were ordered from Integrated DNA Technologies or Genewiz. All plasmids were verified using Sanger sequencing from Genewiz. Tandem repeats were avoided to prevent recombination after transformation and thus did not observe instability of strains or plasmids.

Promoters, operators and tags (P_{BBa_J23100}, P_{lacUV5}, P_{trc}, lacO, ssrA tags) were inserted by Gibson assembly, using extended-length primers with homology arms. Plasmids included pDusk and pDawn (Addgene 43795 and 43796, respectively), Plasmid pMevT, used for mevalonate production (Addgene 17815), Plasmids pSA65 and pSA69, used for isobutanol production.

To construct these OptoLAC circuits, deletion of lacI was performed using the Datsenko-Wanner method, using primers with 70 base pairs of homology to the promoter and terminator regions of the targeted gene. These primers were used to amplify the kanamycin resistance marker flanked by flippase recognition target (FRT) sites from pKD4, or the spectinomycin resistance marker from pYTK9156. Cells were made electrocompetent and electroporated. FRT-flanked resistance markers were cured using flippase (FLP) recombinase from pCP2058. Gene deletions were genotyped by sequencing PCR products amplified from purified genomic DNA using primers flanking the region of deletion.

Strain MG1655 ΔlacI::FRT-KanR-FRTwas obtained as a starting point. From this strain, the kanamycin resistance marker remaining from lacI deletion was removed through FLP-FRT recombination. Hereafter, this strain is referred to as OptoMG (EMAL52). Chemically competent OptoMG was transformed with pDawn controlling lacI (pMAL288) and a reporter plasmid containing P_{T5-lacO}-GFP (pMAL292) to make EMAL57. Three different SsrA tags with progressively weaker degradation strengths (based on the last three residues) were added to the C terminus of LacI: LAA (pMAL294/OptoLAC1), AAV (pMAL296/OptoLAC2) and ASV (pMAL300). OptoMG was transformed with pMAL292 and each of these plasmids to make EMAL68, EMAL69 and EMAL71, respectively.

OptoMG was transformed with pMAL292 and pMAL630 to make EMAL230. The full sequences for OptoLAC1 [SEQ ID NO.: 2], OptoLAC2 [SEQ ID NO.: 3], and OptoLAC3 [SEQ ID NO.: 4] are shown as including several sequences as described previously. For example, the full sequence of OptoLAC1 includes bases for YF1-FixJ (bases 1-1757), a PlacIQ promoter (bases 1758-1835), a P_{FixK2} promoter (bases 2130-2395); cI-LVA (bases 2402-3151); a T_{B0015} terminator (bases 3188-3316); a P_{R} promoter (bases 3325-3408); lacI_ssrA-(LAA) (bases 3415-4530); and a T_{T7} terminator (bases 4619-4666).

As controls for GFP measurement, OptoMG was transformed with pMAL630 (OptoLAC3) and pMAL207 (Empty vector), to produce EMAL229. This strain served as a negative control for subtraction of cell autofluorescence. In addition, OptoMG was transformed with pMAL630 (OptoLAC3) and pMAL301 (PBBA-GFP), to produce EMAL231. This strain expresses GFP constitutively, serving as a positive control to check for photobleaching of GFP under blue light.

Growing EMAL57 in either light or dark conditions results in no significant difference in GFP expression and less than 1% of the constitutive promoter P_{BBa_J23100} (P_{BBA}), indicating that pDawn control of lacI expression requires further engineering to achieve robust optogenetic control of gene expression.

Single colonies from LB+ antibiotic agar plates were inoculated into liquid media and grown in 96-well (USA Scientific Item CC7672-7596) or 24-well (USA Scientific Item CC7672-7524) plates. Strains were grown in a New Brunswick Innova 4000 or Scientific Industries Genie Temp-Shaker 300 (SI-G1600) incubator shaker set to 37° C. and shaken at 200 r.p.m. (19-mm orbital diameter). K strains were cultured in M9 minimal salts medium supplemented with 0.2% wt/vol casamino acids (Bio Basic), K3 trace metal mixture52, 2% wt/vol (20 gl−1) glucose, and appropriate antibiotics (using the previously specified concentrations), unless stated otherwise. B strains were cultured in LB medium (Miller) supplemented with appropriate antibiotics (using the previously specified concentrations). Light-sensitive strains were grown under blue light to keep expression of proteins or metabolic pathways repressed. To stimulate cells with blue (465 nm) light, LED panels (HQRP New Square 12-inch Grow Light Blue LED 14 W) were placed above the culture such that the light intensity was between 80 and 110 μmol m-2 s−1 as measured using a Quantum meter (Apogee Instruments, Model MQ-510), which corresponds to placing the LED panels ˜30 cm from the cultures. To control the light duty cycles, the LED panels were regulated with a Nearpow Multifunctional Infinite Loop Programmable Plug-in Digital Timer Switch.

To measure cell concentration, OD measurements were taken at 600 nm (OD600), using medium (exposed to the same light and incubation conditions as the bacteria cultures) as the blank. Measurements were taken using a TECAN plate reader (infinite M200PRO, data stored in Microsoft Excel) or an Eppendorf spectrophotometer (BioSpectrometer basic) with a microvolume measuring cell (Eppendorf μCuvette G1.0), using samples diluted to a range of OD600 between 0.1 and 1.0.

Super-folder GFP (GFP) expression was quantified by flow cytometry using a BD LSR II flow cytometer (BD Biosciences) and BD FacsDiva 8.0.2 software, with an excitation wavelength of 488 nm and emission wavelength of 530 nm. The gating used in these analyses was defined to include positive (EMAL231) and negative (EMAL229) control cells based on GFP fluorescence, but exclude particles that were either too small or too large to be single living bacterial cells, based on side scatter (SSC-A) versus forward scatter (FSC-A) plots. Median fluorescence values were determined from 10,000 single-cell events.

Single-cell events comprised at least 95% of total events for all samples. The fluorescence data were normalized against the background fluorescence from cells lacking GFP (EMAL229) to account for potential light bleaching and cell autofluorescence. All fluorescence measurements were either taken at the end of the experiments or on aliquots taken from experimental cultures so that potential activation of YF1-FixJ by the light used to excite GFP did not affect the experiments or results. Because the superfolder GFP variant that was used in these examples folds in less than 5 min (ref 53), maturation time should not be a contributing factor in these fluorescence measurements.

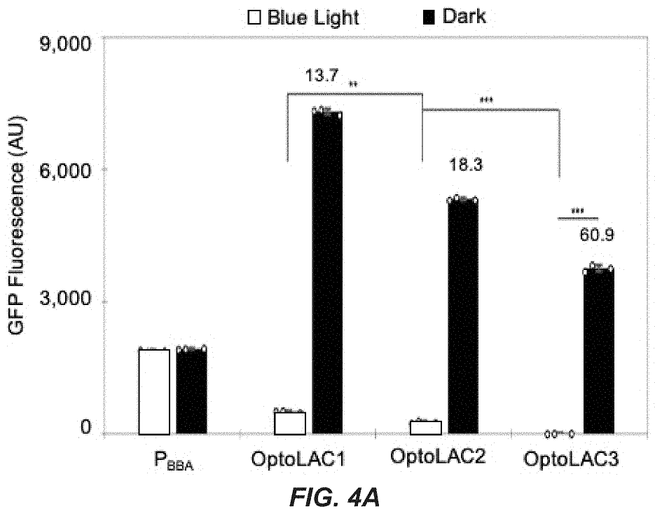

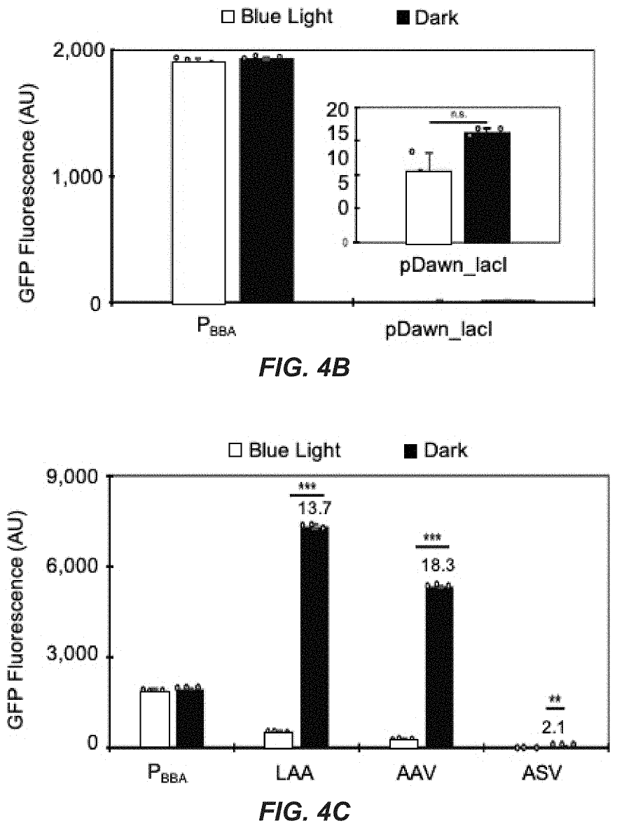

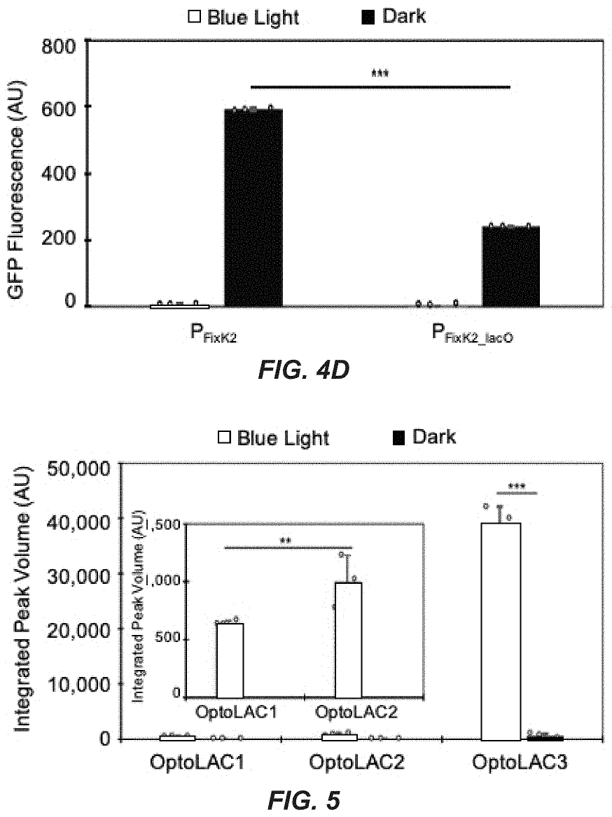

The poor induction and overall low levels of GFP expression in EMAL57 suggest that levels of LacI in this strain are too high, regardless of cI activity. To address this, the half-life of LacI was reduced by fusing C-terminal degradation tags from the 10Sa transfer-messenger RNA (SsrA) of different strength based on the last three amino acids: SsrA-LAA (pMAL294), SsrA-AAV (pMAL296) or SsrA-ASV (pMAL300). Two of the three resulting strains (EMAL68, EMAL69 and EMAL71) show darkness-induced GFP expression levels that exceed constitutive expression from PBBA. Using the strongest SsrA-LAA tag results in the highest GFP expression in the dark. However, the SsrA-AAV tag offers tighter control of gene expression under blue light, probably due to higher LacI-mediated repression. This example continues, using LacI fusions to SsrA-LAA and SsrA-AAV, naming them OptoLAC1 (3.78-fold higher expression than PBBA, with 13.7-fold induction) and OptoLAC2 (2.75-fold higher expression than PBBA, with 18.3-fold induction), respectively (FIG. 4A).

Although fusing LacI to degradation tags improves GFP expression in the dark, it also increases basal expression in the light (FIG. 4A). To reduce the leakiness of OptoLAC2, a lacO operator sequence downstream of the P_{FixK2} promoter controlling cI expression:

SEQ ID NO.: 1
ACGCCCGTGATCCTGATCACCGGCTATCCGGACGAAAACATCTCGA
CCCGGGCCGCCGAGGCCGGCGTAAAAGACGTGGTTTTGAAGCCGCT
TCTCGACGAAAACCTGCTCAAGCGTATCCGCCGCGCCATCCAGGAC
CGGCCTCGGGCATGACCTACGGGGTTCTACGTAAGGCACCCCCCTT
AAGATATCGCTCGAAATTTTCGAACCTCCCGATACCGCGTACCAAT
GCGTCATCACAACGGAGTTGTGAGCGGATAACAAACTAGTAAAGAG
GAGAAA.

Note the lacO sequence (bases 248-264) is underlined, the last 12 bases being the ribosome binding site (RBS).

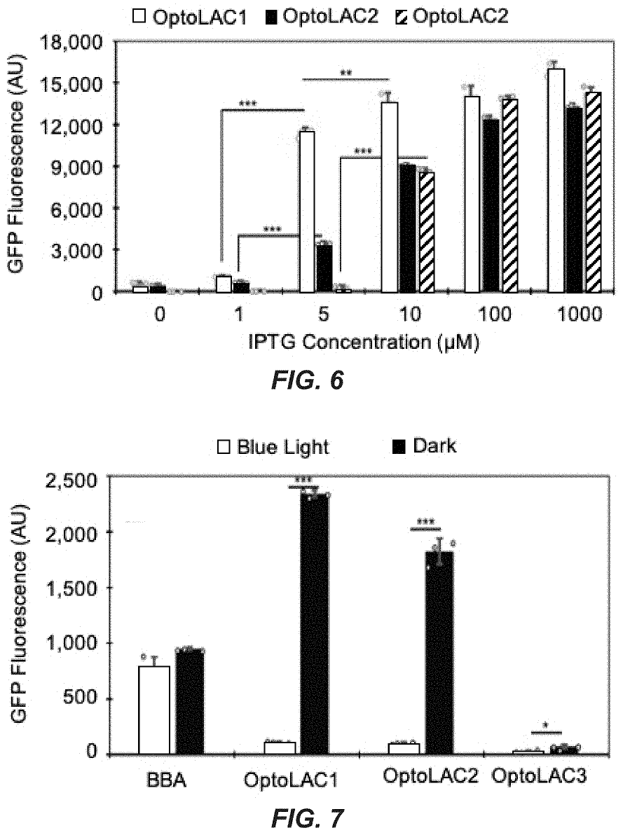

With this design, LacI represses transcription of both the gene of interest (GOI; for example, GFP) and the cI repressor that controls its own expression, creating a positive feedback loop that tightens circuit repression (FIG. 2C). This additional circuit, OptoLAC3 (pMAL630), shows lower GFP expression (strain EMAL230) in blue light than OptoLAC1 or OptoLAC2 (FIG. 4A). However, inserting a lacO sequence also weakens P_{FixK2}, which lowers cI-mediated repression of lacI in the dark, and maximal GFP expression by OptoLAC3. Quantitative western blot analysis confirmed that the positive feedback loop in OptoLAC3 increases the expression of LacI in the light by as much as 60.7- and 53.1-fold relative to OptoLAC1 and OptoLAC2, respectively (FIG. 5). Unlike OptoLAC1 and OptoLAC2, LacI levels in OptoLAC3 remain detectable even after 8 h in the dark (yet 66 times lower than in the light), explaining the lower maximal expression achieved with OptoLAC3. Nevertheless, OptoLAC3 achieves a 60.9-fold induction of gene expression in the dark, which is the highest of the OptoLAC circuits, and still twice the expression achieved from PBBA (FIG. 4A). All three OptoLAC circuits remain IPTG-inducible (FIG. 6), and OptoLAC1 and OptoLAC2 are functional at 37° C. (FIG. 4A) as well as 18° C. (FIG. 7). OptoLAC1, 2 and 3 represent the first suite of optogenetic circuits that harness the lac operon to control gene expression in E. coli with light.

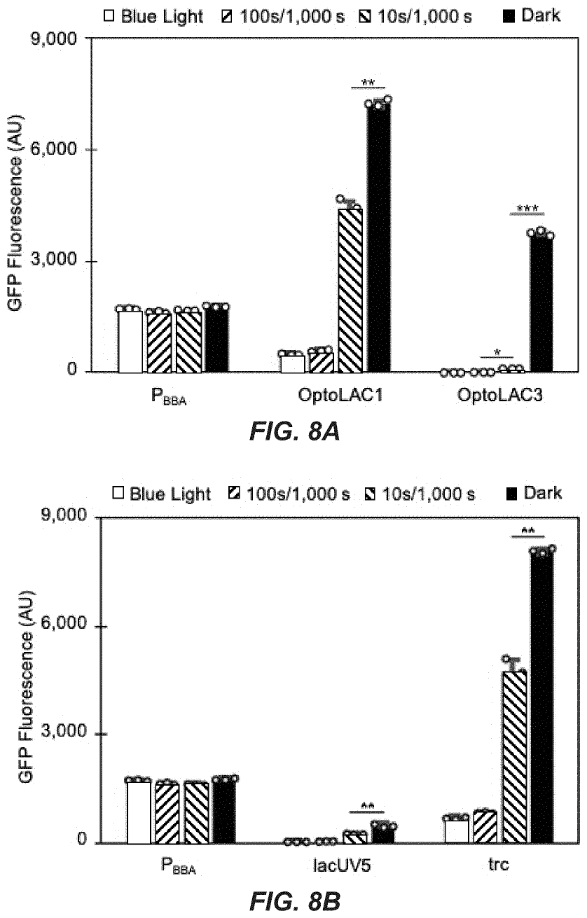

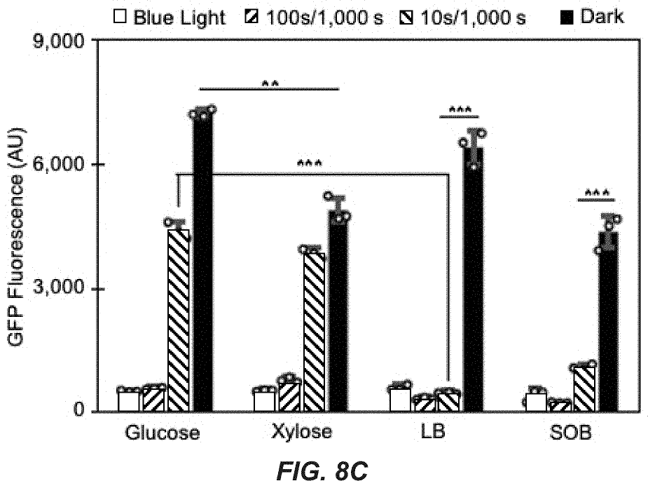

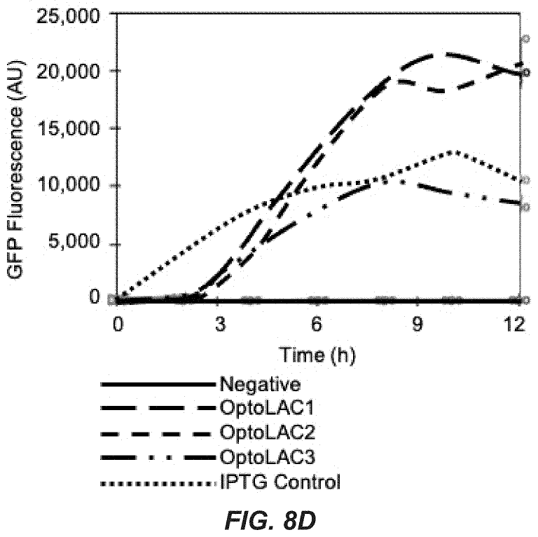

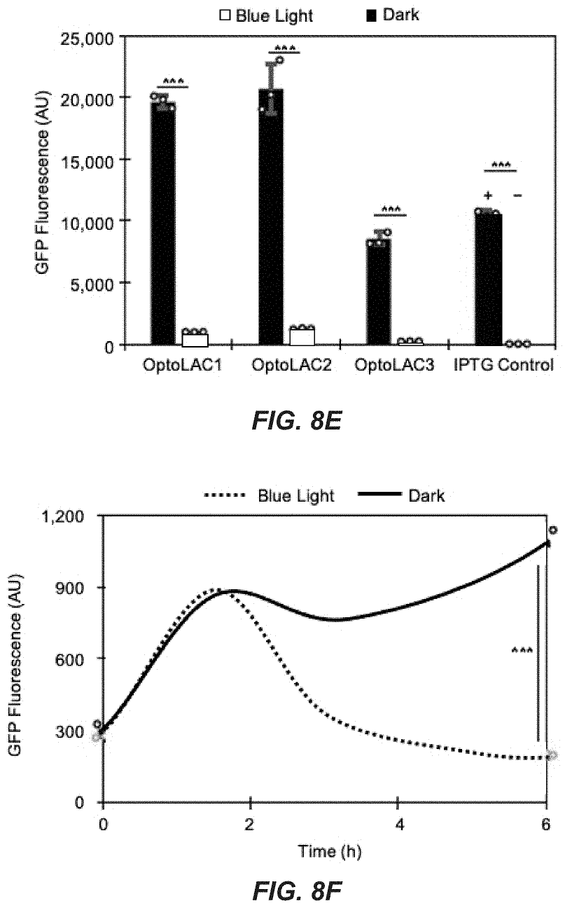

To test the tunability of the OptoLAC circuits, strains containing OptoLAC1 (EMAL68) or OptoLAC3 (EMAL230) were grown under different duty cycles of light. With 1% of light exposure (10 s of light per 1,000 s), OptoLAC1 achieves 61% of maximal expression, and only 10% of light is enough to reach 99% of maximum repression (FIG. 8A)).

To evaluate the response of the circuits to blue light, 1-ml cultures of EMAL57, EMAL68, EMAL69, EMAL71, EMAL229, EMAL230 and EMAL231 were inoculated and grown overnight in M9 medium+2% glucose+ampicillin+kanamycin at 37° C. and 200 r.p.m. under constant blue light to avoid premature transcription of GFP. The next day, the cultures were back-diluted into the same medium to OD_{600=0.01} in 150-μl triplicates into separate 96-well plates and grew the cultures for 8 h under blue light or in the dark (by wrapping the plate in aluminum foil). For light dose-response analysis, additional conditions of light were tested by applying light pulses of 10 s on/990 s off and 100 s on/900 s off. To evaluate the response to light intensity, a rheostat (TerraBloom, VFSC) was used to adjust the panels to different light intensities, while keeping them at the same distance (˜30 cm) from the cell cultures as in previous experiments. After growing for 8 h, 1 μl from each well was diluted into separate wells containing 199 μl of ice-cold phosphate buffered saline (PBS; Corning Life Sciences), kept on ice, and taken for flow cytometry analysis.

To characterize the induction of GFP expression from OptoLAC circuits in OptoMG strains with IPTG under blue light, 1-ml cultures of EMAL229, EMAL68, EMAL69 and EMAL230 were inoculated and grown overnight (as above). The next day, the cultures were back-diluted to OD_{600=0.01} in 150-μl triplicates into separate 96-well plates, added IPTG to a final concentration of 0 μM, 1 μM, 5 μM, 10 μM, 100 μM or 1 mM, and grew the cultures for 8 h under blue light. Samples were then prepared as explained above and taken for flow cytometry analysis.

To analyze circuit performance at lower temperatures, 1-ml cultures of EMAL229, EMAL231, EMAL68, EMAL69 and EMAL230 were inoculated and grown overnight (as above). The next day, the cultures were back-diluted to OD₆₀₀=0.01 in 150 μl triplicates into separate 96-well plates and grew the cultures for 48 h under blue light or in the dark (by wrapping the plate in aluminum foil) at 18° C. Samples were then taken for flow cytometry analysis (as above).

OptoLAC3 is even more sensitive than OptoLAC1, achieving full repression under only 1% of light duty cycle (FIG. 8A). Although both circuits should, in principle, be controllable by the intensity of continuous illumination, their high sensitivity to even the lowest intensities one can practically provide (e.g., 5 μmol m-2s-1) makes them more easily tunable using light duty cycles. The differences in light sensitivities between circuits may be useful for different applications: for example, OptoLAC1 is best suited to maintain intermediate levels of expression, while OptoLAC3 can better achieve full gene repression when light penetration is limited.

To test whether OptoLAC circuits can control other lacO-containing promoters besides the P_{T5-lacO} promoter, OptoLAC1 was used to control GFP expression from P_{lacUV5} and P_{trc} with light.

To test the performance of the circuits with different IPTG-inducible promoters, chemically competent OptoMG was transformed with pMAL294 (OptoLAC1) and one of the following two plasmids: a minimal-copy reporter plasmid containing PlacUV5-GFP (pMAL302) or Ptrc-GFP (pMAL303). These strains were named EMAL267 and EMAL268, respectively. 1-ml overnight cultures of EMAL229, EMAL231, EMAL267 and EMAL268 were inoculated (as above). The next day, the cultures were back-diluted to OD₆₀₀=0.01 in 150-μl triplicates into separate 96-well plates and grew them for 8 h under blue light (pulses of 10 s on/990 s off and 100 s on/900 s off) or in the dark (by wrapping the plate in aluminum foil). Samples were then taken for flow cytometry analysis (as above).

Both promoters show light-tunable repression with varying degrees of maximal gene expression in the dark according to the core promoter strength (FIG. 8B). This finding demonstrates that the OptoLAC circuits can be used to control different IPTG-inducible promoters, which facilitates the retrofitting of many existing systems with light controls.

Inducible promoters are frequently used for dynamic control in metabolic engineering to produce fuels and chemicals from renewable sources, such as glucose or xylose. In addition, different applications require minimal or rich media such as LB medium or super optimal broth (SOB) medium. OptoLAC1 was tested in M9 minimal salts medium with glucose or xylose, as well as in LB or SOB media, and found it to be effective at controlling gene expression with light in all media tested (FIG. 8C).

To test different carbon sources, 1-ml overnight cultures of EMAL229, EMAL231 and EMAL68 were inoculated in M9 medium+20 gl-1 glucose or xylose, LB medium (Miller) or SOB medium (at 37° C. and 200 r.p.m.). The next day, the cultures were back-diluted to OD₆₀₀=0.01 in 150-μl triplicates into separate 96-well plates, and grew the cultures for 8 h under full blue light (light pulses of 10 s on/990 s off or 100 s on/900 s off) or in the dark (by wrapping the plate in aluminum foil). Samples were then taken for flow cytometry analysis (as above). To quantify differences in LacI protein levels for OptoLAC1, OptoLAC2 and OptoLAC3, 1-ml overnight cultures of EMAL68, EMAL69 and EMAL230 were innoculated (as above). The next day, the cultures were back-diluted to OD₆₀₀=0.01 in 1-ml triplicates into separate 24-well plates and grew the cultures for 8 h under blue light or in the dark (by wrapping the plate in aluminum foil). The OD₆₀₀ of each sample was measured before harvesting the full 1-ml culture for western blot analysis.

However, OptoLAC1 is more light-sensitive in rich media than in minimal media, while its maximal gene expression in the dark is greater in glucose than in xylose. Without being limited to theory, it is believed the decreased sensitivity of OptoLAC1 in M9 medium may be due to stress response in minimal media, which could lead to higher rates of LacI degradation and FixJ phosphorylation, as proteolysis and increased phosphorylation of two-component systems are associated with this response.

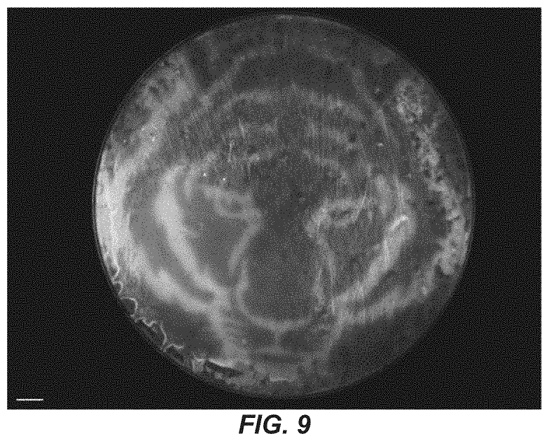

OptoLAC circuits can control gene expression not only temporally in liquid media, but also spatially in solid media by shielding specific sections of an LB agar plate from blue light (FIG. 9).

To test spatial control of GFP expression, a 1-ml overnight culture of EMAL68 was inoculated (as above). The next day, 500 μl of culture was spread onto a 150 mm Å˜15 mm LB medium+kanamycin+ampicillin agar plate (Laboratory Disposable Products, catalog no. 229656-CT) using sterile glass beads. The plate was placed in ambient temperature on top of a black cloth and 35 cm underneath a projector (Epson H764A) displaying an image of a tiger (Drawing Work). The set-up was covered in black cloth to prevent ambient light contamination. After 16 h, the plate was imaged for GFP fluorescence using a Bio-Rad ChemiDoc MP Imaging System with Image Lab software, with Blue Epi illumination using a 530/28 filter (filter 4).

Therefore, OptoLAC circuits provide spatiotemporal control over gene expression in different media for the potential benefit of many applications.

OptoLAC circuits were compared with IPTG, the current gold standard method to induce gene expression in E. coli. Two-phase time-course experiments were performed to compare these systems, implementing a growth phase (light/no IPTG) followed by a production phase (darkness/IPTG).

Expression of GFP from OptoLAC circuits is detectable 2-4 h after inducing with darkness, whereas IPTG induction responds more rapidly with stronger GFP expression by the second hour after induction (FIG. 8D). This is consistent with LacI degradation, required to activate gene expression in OptoLAC circuits, being inherently slower than allosteric inhibition of LacI by IPTG. However, it still takes 10 h to reach full induction of GFP with IPTG, at which point the levels of expression achieved with OptoLAC1 and OptoLAC2 are higher by 63% and 41%, respectively. GFP expression obtained with IPTG induction is matched by OptoLAC1 and OptoLAC2˜4-5 h after induction, and by OptoLAC3 after 8 h. Importantly, exposing the cultures containing OptoLAC circuits to blue light for the entire time course keeps GFP expression repressed (FIG. 8E). Moreover, while strains containing OptoLAC circuits have a slightly increased lag phase compared to the IPTG-induced control, they show comparable growth rates in exponential phase and final biomass yields. Despite their initial delay in induction, OptoLAC circuits eventually catch up and even exceed the induction levels achieved with IPTG, with minimal impact on cell growth, suggesting that the inhibition of LacI by IPTG is incomplete and its transcriptional repression is ultimately more effective.

A major advantage of optogenetic controls over chemical induction is reversibility, as turning lights on and off is easier and faster than changing media. To explore this capability, the on-to-off kinetics of OptoLAC circuits were tested using an unstable PTs-co-GFP reporter fused to a C-terminal SsrA (AAV) tag (pMAL897) and inducing cultures in the dark for 3 h before turning on the lights for 6 h.

An IPTG-inducible control strain was prepared that contained constitutively expressed lacI from a plasmid of the same copy number to the OptoLAC circuits by transforming OptoMG with pMAL292 and pET28a, creating strain EMAL77. 1-ml overnight cultures of EMAL229, EMAL68, EMAL69, EMAL230 and EMAL77 were inoculated (as above). The next day, the cultures were back-diluted to OD₆₀₀=0.01 in 1-ml triplicates into eight separate 24-well plates and grew the cultures for ˜4 h under blue light, at which point the cultures reached OD₆₀₀=0.9. At this point, samples were collected from one plate (corresponding to t=0 h). For strains EMAL229, EMAL68, EMAL69 and EMAL230, six of the remaining seven plates were induced by wrapping them in aluminum foil. EMAL77 was induced by adding 1 mM IPTG to six of the remaining seven plates, to a final concentration of 1 mM. The final (eighth) plate was left in blue light (for EMAL77, without IPTG) for 12 h as a control. Every 2 h up to 12 h after induction, one plate was uncovered to measure the levels of GFP expression (and then discarded), by diluting 1 μl of culture into separate wells containing 199 μl of ice-cold PBS (Corning Life Sciences) and keeping on ice for flow cytometry analysis.

To explore the on-to-off kinetics, chemically competent OptoMG was transformed with either OptoLAC1, OptoLAC2 or OptoLAC3 and a minimal-copy reporter plasmid containing destabilized PT5-lacO-GFP-SsrA-AAV (pMAL897). These strains were named EMAL343, EMAL344 and EMAL345, respectively. 1-ml overnight cultures of EMAL229, EMAL343, EMAL344 and EMAL345 were inoculated (as above). The next day, the cultures were back-diluted to OD₆₀₀=0.1 in 1-ml triplicates into nine separate 24-well plates and grew the cultures for ˜3 h in darkness (by wrapping them in aluminum foil), at which point the cultures reached OD₆₀₀=0.9. At this point, samples were collected from one plate (corresponding to t=0 h). Four of the remaining eight plates were then repressed (uninduced) by removing them from foil and culturing them under constant blue light. The remaining four plates were left in darkness as controls. Every 1.5 h up to 6 h after induction, one plate from each condition was uncovered to measure the levels of GFP expression (and then discarded), by diluting 1 μl of culture into separate wells of a 96-well plate containing 199 μl of ice-cold PBS (Corning Life Sciences), and kept on ice for flow cytometry analysis.

OptoLAC1 (EMAL343) exhibits a 1.5-h delay before GFP levels decrease, with an ˜1-h half-life signal decay thereafter (FIG. 8F). The timescale of these experiments does not permit sufficient GFP-SsrA (AAV) expression with the weaker OptoLAC2 and OptoLAC3 to carry out the same measurements, although the observed trends are as expected. Without being held to theory, it is believed the ON to OFF kinetics (dark to light) is determined by the rates of cI degradation (or mitotic dilution), lacI accumulation, and GFP degradation (or mitotic dilution). While using a destabilized GFP helps reduce this last time delay, it also reduces the amount of GFP accumulation during the ON phase of the experiment, which prevents us from measuring the off kinetics of the weaker OptoLAC2 and OptoLAC3 circuits in the short timescales of these batch fermentations. Nevertheless, the strongest OptoLAC1 achieves enough GFP accumulation to measure the OFF kinetics in short 9-hour fermentations, demonstrating the potential of these circuits for reversibility.

Although OptoLAC circuits are specifically designed for efficient gene induction in the dark, at least OptoLAC1 offers reversibility in short fermentations (9 h), which cannot be replicated with IPTG induction.

### Optogenetic Control of Isobutanol Production

Given the potential of optogenetics for metabolic engineering, OptoLAC circuits were tested in light-controlled fermentations for chemical production.

First, the circuits were applied to control the biosynthesis of the advanced biofuel isobutanol. This included two sequences, one placing BsalsS, ilvC, and ilvD under a P_{L-lacO1} promoter, and Llkivd and LladhA under a separate P_{L-labO1} promoter, placing an entire biosynthesis pathway that converts glucose to isobutanol under those lacO operator-controlled promoters. Previously developed plasmids containing an IPTG-inducible isobutanol pathway to the optogenetic platform were adapted. This generated strains that control isobutanol production using OptoLAC1 (EMAL199), OptoLAC2 (EMAL200), OptoLAC3 (EMAL239) or IPTG (EMAL201). The optical density at 600 nm (OD600) at which one switches fermentations controlled with OptoLAC circuits from light to darkness (ρₛ) has a substantial impact on final isobutanol titers.

To construct the isobutanol-producing strains, chemically competent OptoMG cells were transformed with either OptoLAC1, OptoLAC2 or OptoLAC3 and a plasmid that combines all the genes from pSA65 and pSA69 in a single vector with a p15A origin of replication and β-lactamase resistance marker (pMAL534), generating strains EMAL199, EMAL200 and EMAL239, respectively. A light-insensitive IPTG-inducible control strain was made by co-transforming chemically competent OptoMG with pMAL534 and pET28a, generating EMAL201.

These strains (EMAL199, EMAL200, EMAL239 and EMAL201) express (from the P_{L-lacO1} promoter) three enzymes that convert pyruvate to 2-ketoisovalerate: acetolactate synthase from Bacillus subtilis (BsalsS), acetohydroxyacid reductoisomerase (ilvC) and dihydroxyacid dehydratase (ilvD). They also express, from another P_{L-lacO1} promoter, two enzymes that convert 2-ketoisovalerate into isobutanol: 2-ketoacid decarboxylase from Lactococcus lactis (Llkivd) and alcohol dehydrogenase from Lactococcus lactis (LladhA).

The transformants were plated on LB medium+kanamycin+ampicillin agar and colonies were grown under blue light to avoid negative selection due to the potential pathway expression. Four colonies from each strain were screened for isobutanol production. Each colony was used to inoculate 1 ml of M9+2% glucose+kanamycin+carbenicillin medium, grown overnight at 37° C. and 200 r.p.m. under blue light. The next day, each culture was back-diluted into the same medium to an OD600 of 0.01 and grown for 4 h at 37° C. at 200 r.p.m. under blue light. The plates were then sealed with Nunc Sealing Tape (Thermo Scientific) and wrapped in aluminum foil; for the control strain EMAL201, IPTG was added to 1 mM before sealing and wrapping. No holes were poked in the sealing tape to prevent evaporation of isobutanol. The strains were fermented in the dark at 30° C. at 200 r.p.m. for 72 h, after which samples were prepared for HPLC analysis as described above. The highest-producing colonies were selected for subsequent optimization.

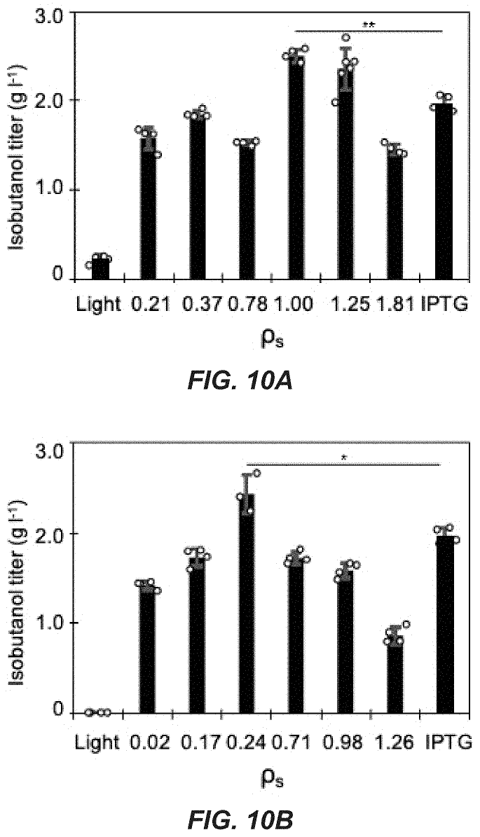

To find the optimal value of ps for isobutanol production, 1-ml overnight cultures of each strain were back-diluted to different OD600 values (ranging from 0.001 to 0.1) in 1-ml cultures of the same media described above (in quadruplicates). The different dilutions were grown for 4 h, reaching different OD600 values, at which time cultures were switched from light to dark conditions (see FIGS. 10A, 10B), or induced with IPTG. As controls, each strain was grown under continuous blue light (EMAL199, EMAL200, EMAL239) or with no IPTG added (EMAL201).

The fermentation procedure included, for isobutanol production, transferring 700 μl of cell culture to a 2-ml microcentrifuge tube and centrifuged at 17,000 r.c.f. for 45 min at 4° C. in a benchtop centrifuge (Eppendorf Centrifuge 5424) to obtain cell-free supernatant, then 250 μl of the supernatant was transferred to an HPLC vial for analysis.

Cell-free supernatant samples were analyzed via liquid chromatography (Agilent 1260 Infinity) using an Aminex HPX-87H ion-exchange column (Bio-Rad). The mobile phase was 5 mM sulfuric acid. Samples were run through the column at 55° C. and a flow rate of 0.6 ml min−1. Isobutanol was monitored with a refractive index detector (RID, Agilent G1362A). To determine their concentration, the peak areas were measured using Agilent OpenLab CDS Chemstation software and compared to those of their own standard solutions for quantification.

Those induced at their optimal ps values achieve as much as 27% higher isobutanol titers (2.5±0.1 gl−1 and 2.4±0.2 gl−1 with OptoLAC1 and OptoLAC3, respectively) than fermentations induced with IPTG at their optimal OD600 of induction (2.0±0.1 gl−1, OD600=1.4) (FIGS. 10A, 10B). The optimal ρₛ values differ substantially between OptoLAC1 (ρₛ=1.0) and OptoLAC3 (ρs=0.24).

Thus, OptoLAC circuits can replace IPTG with darkness as an inducing agent in E. coli fermentations for chemical production.

### Optogenetic Control of Mevalonate Production

To test whether the disclosed optogenetic circuits can be applied to other metabolic pathways, they were used to produce mevalonate, an important terpenoid precursor.

Again, a previously described IPTG-inducible plasmid was adapted, containing the mevalonate pathway. Strains were constructed in which mevalonate biosynthesis is controlled with light using OptoLAC1 (EMAL208), OptoLAC2 (EMAL209) or OptoLAC3 (EMAL235), as well as an IPTG-induced control (EMAL135).

To construct these mevalonate-producing strains, chemically competent OptoMG cells with either OptoLAC1, OptoLAC2 or OptoLAC3 and a modified version of pMevT18 were transformed, in which P_{lac} was replaced with P_{lacUV5} (pMAL487), generating strains EMAL208, EMAL209 and EMAL235, respectively. A light-insensitive IPTG-inducible control strain was made by transforming chemically competent OptoMG cells with pMAL487 and pET28a, generating EMAL135. These strains (EMAL208, EMAL209, EMAL235 and EMAL135) express, from a single operon, the first three enzymes of the mevalonate pathway, which convert acetyl-CoA into mevalonate: acetoacetyl-CoA thiolase (atoB) from E. coli, 3-hydroxy-3-methylglutaryl-CoA synthase (HMGS or ERG13) from S. cerevisiae, and a truncated version of 3-hydroxy-3-methylglutaryl-CoA reductase (tHMGR) from S. cerevisiae. The transformants were plated on LB+kanamycin+chloramphenicol agar and colonies were grown under blue light to avoid potential negative selection due to pathway expression. Four colonies from each strain were screened for mevalonate production. Each colony was used to inoculate 1 ml of M9+2% glucose+chloramphenicol+kanamycin medium, grown in a 24-well plate overnight at 37° C. and 200 r.p.m. under blue light. The next day, each culture was back-diluted into the same medium to an OD600 of 0.01 and grown for 4 h at 37° C. at 200 r.p.m. under blue light. The plates were then sealed with Nunc Sealing Tape (Thermo Scientific) and wrapped in aluminum foil; for the control strain EMAL135, IPTG was added to 1 mM before sealing and wrapping. A single hole (using a sterile syringe needle) was poked in the center of the sealing tape covering each sample well to allow limited aeration.

The strains were fermented in the dark at 37° C. at 200 r.p.m. for 72 h, after which samples were prepared for HPLC analysis: 560 μl of cell culture was mixed with 140 μl, of 0.5 M HCl in a 1.5-ml microcentrifuge tube and vortexed at high speed for 1 min to convert mevalonate to (±)-mevalonolactone. The mixture was then centrifuged at 17,000 r.c.f. for 45 min at 4° C. in a benchtop centrifuge (Eppendorf Centrifuge 5424) to obtain cell-free supernatant. Then, 250 μl, of this supernatant was transferred to an HPLC vial for analysis.

The highest-producing colonies were selected for subsequent optimization. To find the optimal value of ρs for mevalonate production, 1-ml overnight cultures of each strain were back-diluted to different OD600 values (ranging from 0.001 to 0.1) in 1-ml cultures of the same media as described above (in quadruplicates).

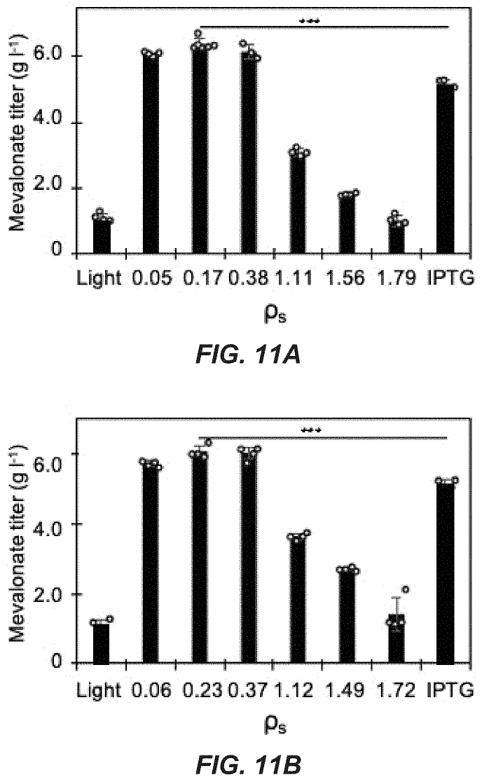

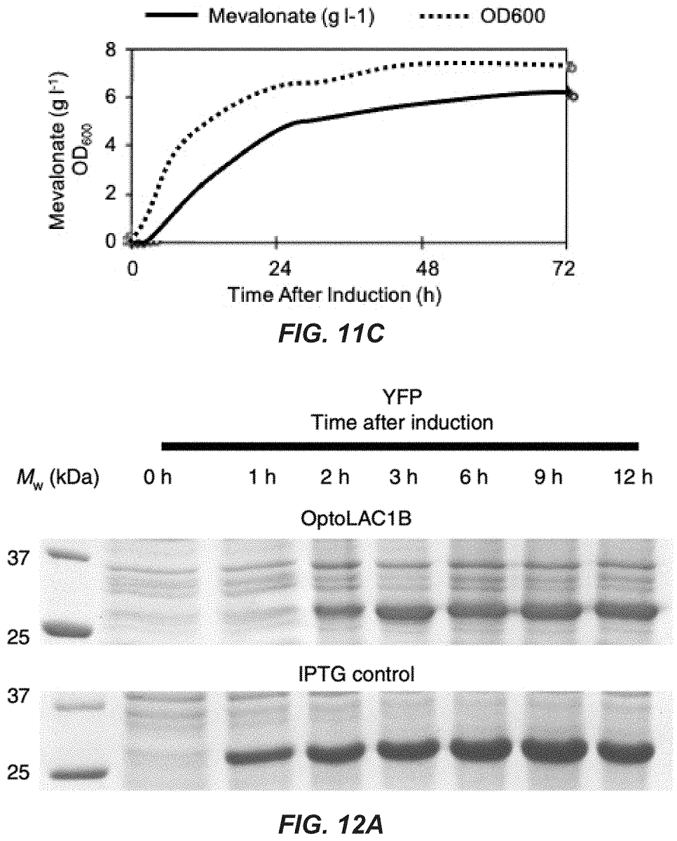

The different dilutions were grown for 4 h, reaching different OD600 values, at which time cultures were switched from light to dark conditions (see FIGS. 11A, 11B) or induced with IPTG (Extended Data FIG. 8b). As controls, each strain was grown under continuous blue light (EMAL208, EMAL209, EMAL235) or with no IPTG added (EMAL135). The same fermentation procedure described above was then followed.

OptoLAC1 and OptoLAC2 achieve the highest titers of 6.4±0.2 gl−1 and 6.1±0.2 gl−1, respectively, at similar ps values (ρₛ=0.17-0.23), which exceed the titer obtained with IPTG at its optimal OD600 of induction (5.2±0.1 gl−1, OD600=0.06) by as much as 24% (FIGS. 11A, 11B). The low optimal ps values and high performance of OptoLAC1 and OptoLAC2 suggest that early and high maximal induction are key to optimizing mevalonate production.

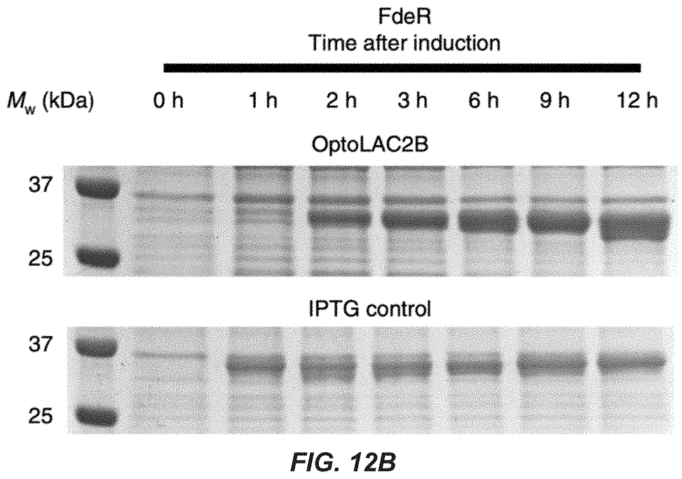

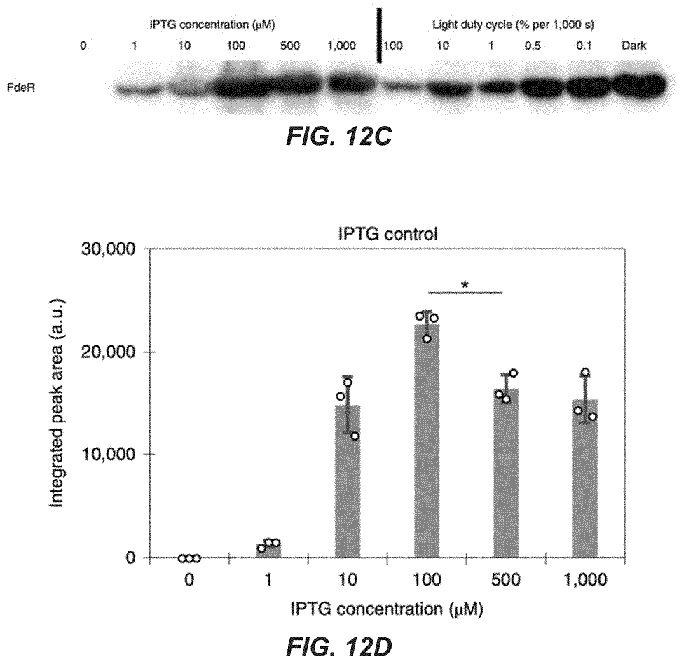

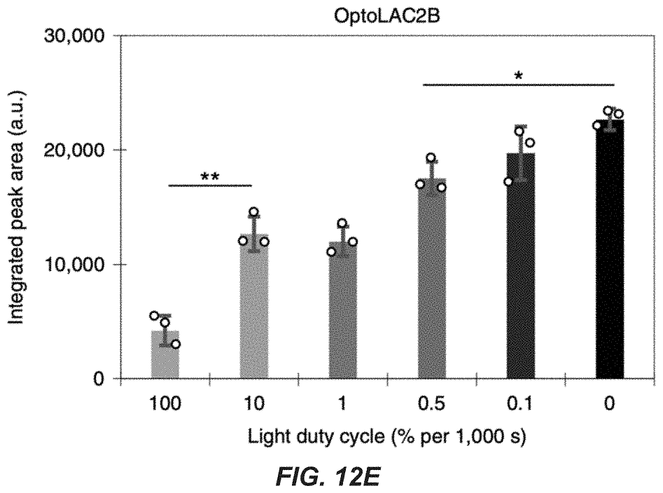

Without being held to theory, this is probably due to differences in circuit strength. The optogenetic circuits match or exceed the production levels achieved with IPTG induction. Despite the initial delay, OptoLAC circuits eventually match or surpass the levels of GFP expression (FIG. 8D) and recombinant protein production (FIGS. 12A, 12B) obtained with IPTG. Additionally, OptoLAC circuits can outperform IPTG induction for mevalonate and isobutanol production. While previous studies achieve higher titers for these chemicals by deleting competing pathways and optimizing fermentation conditions, the strains in this study only contain plasmids with biosynthetic pathways, which is enough to demonstrate the functionality of the circuits and, because the control strains are equally engineered, also provide a fair comparison with IPTG induction. These results suggest that, at least in some conditions, cI mediated repression of lacI by OptoLAC circuits is more effective at allowing gene expression from lacO-containing promoters than IPTG is at sustaining full inhibition of LacI. The disclosed suite of OptoLAC circuits provides flexibility to control engineered metabolic pathways. OptoLAC1 and OptoLAC2 are the best at producing mevalonate (6.4±0.2 and 6.1±0.2 g/L, respectively) at relatively low optimal ρₛ values (0.17 and 0.23, respectively). Given mevalonate's low toxicity, inducing at low OD₆₀₀ would be expected to improve production. In contrast, OptoLAC1 and OptoLAC3 are the best at producing isobutanol (2.5±0.1 and 2.4±0.2 g/L, respectively), but at substantially different ρₛ values (1.00 and 0.24, respectively), which reflects the ability of the disclosed circuits to adapt to the relatively higher toxicity of isobutanol. In the case of OptoLAC1, which is stronger but also leakier than OptoLAC3, maximum titers are achieved when inducing at higher OD600, possibly due to the accumulation of toxic product during the growth phase. On the other hand, OptoLAC3 is not as strong as OptoLAC1 but is better at keeping isobutanol biosynthesis tightly repressed, which may explain why maximum isobutanol titers are achieved when inducing at lower OD600. This highlights the benefit of having a flexible suite of OptoLAC circuits with different strengths, sensitivities, and folds of induction, which could make each of them more suitable for different metabolic pathways or biotechnological applications.

OptoLAC circuits can thus control different metabolic pathways with light, improving upon the titers obtained with IPTG induction.

To test the scalability of this approach, EMAL208 (OptoLAC1) was used to produce mevalonate in a light-controlled 2-l bioreactor.

A 5-ml overnight culture of EMAL208 (OptoLAC1 driving mevalonate production) in M9+5% glucose+kanamycin+chloramphenicol under blue light at 37° C. A BioFlo120 system was set up with a 2-l bioreactor (Eppendorf, B120110001) and added 1 l of sterile M9+5% glucose+kanamycin+chloramphenicol. The reactor was set to 37° C., pH 7.0 (which was maintained using a base feed of ammonium hydroxide (Thomas Scientific), and a minimum dissolved oxygen percentage of 20 (maintained by adjusting the agitation rate between 200 and 800 r.p.m. and by injecting air at a flow rate of 0.1-3.0 SLPM (standard litres per minute)). Three blue LED panels were placed in a triangular formation ˜20 cm from the reactor such that the light illuminated ˜76% of the bulk surface area at an intensity of 80-110 μmol m-2 s−1. The reactor was then inoculated to an OD600 of 0.004, and the cells were grown for ˜4 h under blue light until reaching an OD600 of 0.17. The lights were then turned off and the reactor was wrapped in aluminum foil and covered with black cloth. At 8 h, 50 μl of Antifoam 204 (Sigma-Aldrich) was added to prevent foaming. Culture samples of 1 ml were taken while minimizing potential exposure of the culture to ambient light and prepared for HPLC analysis as described above.

For the first 3 h after induction, mevalonate production is undetectable, demonstrating that blue light effectively represses the mevalonate pathway during the growth phase (FIG. 11C). By the sixth hour after induction, mevalonate production increases rapidly, reaching 6.3±0.2 gl−1 after 72 h of induction.

Overall, the results suggest that OptoLAC circuits respond equally to the light penetration achievable in fermentations scaled up at least three orders of magnitude.

Without being held to theory, it is suggested that OptoLAC circuits are designed to be inducible by darkness to preempt potential challenges of light penetration. This feature might have played a role in the successful scale-up experiments in the 2-L bioreactor. However, the circuits are also remarkably sensitive to even the lowest light intensity (5 μmol/m2/s) that could be attained. In addition, all optimal ρₛ values found for chemical or protein production are low (OD₆₀₀<1.5), which makes light penetration less challenging. Thus, inducing gene expression with darkness, the high sensitivity displayed by the disclosed OptoLAC circuits, and availability of several photobioreactor designs bode well for overcoming light penetration hurdles in future scaling up efforts.

### Optogenetic Control of Recombinant Protein Production

To demonstrate the ability of OptoLAC circuits to control LacI-regulated (IPTG-inducible) promoters in other applications, the circuits were tested for light control of recombinant protein production. The platform was adapted to the workhorse B-strain for protein production, BL21 DE3, by deleting its two endogenous copies of lacI, resulting in EMAL255 (hereafter OptoBL). The constitutive copy of lacI was removed from pCri-8b (containing P_{T7}-YFP (yellow fluorescent protein)) to produce plasmid pC85. Finally, OptoLAC1 and OptoLAC2 were reconstructed into a pACYC-derived backbone, resulting in OptoLAC1B (pMAL658) and OptoLAC2B (pMAL659), respectively, which have different origins of replication and are thus compatible with commonly used pET and pCri protein production vectors.

Using these tools, EMAL284 was created which can be induced with darkness using OptoLAC1B to produce. OptoLAC1B keeps YFP tightly repressed under light (t=0 h in FIG. 12A) and effectively induces its production in the dark. Although OptoLAC1B shows an ˜2-h delay in detectable protein production, after 9-12 h of darkness induction, the levels of protein production achieved by OptoLAC1B are comparable to those achieved with an IPTG-induced control (EMAL283; FIG. 12A). The optimal cell density of induction for YFP production (after 9 h) is similar for both IPTG and darkness (OD600=0.1).

Similar to OptoLAC circuits in OptoMG, OptoLAC1B has a minimal impact on OptoBL growth in two-phase fermentations, causing a slight decrease in growth rate compared to the IPTG-induced control, but no significant difference in final biomass yield.

Under non-inducing conditions (light/no IPTG), the impact of OptoLAC1B is smaller than that caused by plasmid pRARE2 in the Rosetta 2 strain. OptoLAC circuits can thus be robustly transferred across different strains and plasmids for different biotechnological applications.

As a second example for recombinant protein production, FdeR, a transcription factor from Herbaspirillum seropedicae was chosen. OptoBL was transformed with OptoLAC1B (pMAL658) or OptoLAC2B (pMAL659), and pMAL887 to produce EMAL335 and EMAL336, respectively. OptoLAC2B displays tighter control over FdeR expression when cells are grown in constant blue light for 12 h. Similar to YFP with OptoLAC1B, FdeR production from OptoLAC2B is tightly controlled before induction (t=0 h in FIG. 12B) and, despite an ˜2-h delay, FdeR production with OptoLAC2B is comparable to that of an IPTG-inducible control (EMAL329) 9-12 h after induction (FIG. 12B). Moreover, FdeR production remains high over a broader range of ρₛ values (OD600=0.2-1.0) compared to cell densities of IPTG induction of EMAL329 (OD600=0.5-1.0).

To provide more detail, to develop strains for protein production, the endogenous copy of lacI was knocked from BL21 DE3 and removed the remaining kanamycin resistance marker left by the knockout (as described above), resulting in EMAL224. The copy of lacI introduced by the DE3 prophage was then deleted, resulting in EMAL255. This strain is referred to as OptoBL. For YFP production, electrocompetent OptoBL cells were transformed with pMAL658 (OptoLAC1B) and pC85 (P_{T7}-YFP), resulting in EMAL284. As an IPTG-inducible control containing constitutive lacI, BL21 DE3 was transformed with pCri-8b21 (P_{T7}-YFP, P_{lacIQ_lacI}), resulting in EMAL283. For FdeR production, electrocompetent OptoBL cells were transformed with one of pMAL658 (OptoLAC1B) or pMAL659 (OptoLAC2B), as well as pMAL887 (P_{T7}-FdeR), resulting in EMAL335 and EMAL336, respectively. As an IPTG-inducible control, BL21 DE3 was transformed with pC9 (PT7-FdeR, P_{lacIQ_lacI}), which contains constitutive lacI, resulting in EMAL329. Transformation agar plates for light-sensitive strains were grown under constant blue light to avoid negative selection due to protein expression.

To determine if these OptoLAC circuits impact cell growth in E. coli B strains (OptoBL), 1-ml overnight cultures of EMAL276, EMAL283, and EMAL284 were inoculated in LB medium and their appropriate selection antibiotics (EMAL284 also in blue light). The next day, the cultures were back-diluted to OD600=0.01 in 1-ml triplicates into two separate 24-well plates. One plate was grown under continuous blue light (for EMAL276 and EMAL283, without IPTG) for 15 h; the other plate was grown under blue light for 4 h, then switched to darkness for 11 h (for EMAL283, without IPTG for 4 h, then with 1 mM IPTG for 11 h). In both plates, additional triplicates of EMAL284 were grown that were induced with 1 mM IPTG after 4 h of growth. OD600 measurements were taken every 2 h from the time of inoculation.

For kinetic analysis of protein production, 1-ml overnight cultures of EMAL283, EMAL329, EMAL284 and EMAL336 were inoculated in LB medium and their appropriate selection antibiotics (EMAL284 and EMAL336 also in blue light). The next day, the cultures were back-diluted to OD600=0.03 in 1-ml cultures into eight separate 24-well plates and the cultures were grown to OD600=0.5 (which took ˜3 h) under blue light. Seven of the plates were switch to the dark by wrapping them in aluminum foil, adding IPTG to a final concentration of 1 mM for EMAL283 and EMAL329. One plate was left in blue light and without IPTG for 12 h as a control. The OD600 of each sample was measured before harvesting the full 1-ml culture at 0 h, 1 h, 2 h, 3 h, 6 h, 9 h and 12 h for analysis.

To find the optimal ps value for protein production, 1-ml overnight cultures of EMAL283, EMAL329, EMAL284 and EMAL336 were inoculated (as above). The next day, the cultures were back-diluted to OD600 values between 0.01 and 0.1 in 1-ml cultures into a 24-well plate and the cultures were grown to OD600 values between 0.1 and 1.8 (which took ˜3 h) under blue light. Protein expression was induced by switching the plate to the dark (wrapping it in aluminum foil) or by adding IPTG to a final concentration of 1 mM for EMAL283 and EMAL329. As controls, cultures were grown that were kept under blue light/without IPTG for the duration of the experiment (non-induced), as well as cultures of EMAL284 and EMAL336 that were induced with 1 mM IPTG after 3 h of growth but kept under blue light. The OD600 of each sample was measured before harvesting the full 1-ml culture at 9 h for analysis.

Thus, both OptoLAC1B and OptoLAC2B can control the production of different recombinant proteins at yields comparable to those achieved with IPTG induction. Easy tunability of gene expression is a potential advantage of optogenetic control over chemical induction. To showcase this capability, YFP and FdeR production induced with different duty cycles of blue light (EMAL284, EMAL336) or different IPTG concentrations (EMAL283, EMAL329) was quantified.

1-ml overnight cultures of EMAL283, EMAL329, EMAL284 and EMAL336 were inoculated (as above). The next day, the cultures were back-diluted to OD600=0.01 for YFP or OD600=0.03 for FdeR in 1-ml cultures into six separate 24-well plates and grew the cultures to OD600=0.1 for YFP or OD600=0.5 for FdeR (which took ˜3 h) under blue light. The plates were moved to six separate light conditions: full blue light; pulses of 1 s on/999 s off, 5 s on/995 s off, 10 s on/990 s off, 100 s on/900 s off; or in the dark (by wrapping the plate in aluminum foil). For EMAL283 and EMAL329, the cultures were back-diluted to OD600=0.01 for YFP or OD600=0.03 for FdeR in 1-ml cultures into a 24-well plate and the cultures were grown to OD600=0.1 for YFP or OD600=0.5 for FdeR (which took ˜3 h). IPTG was then added to final concentrations of 0 μM, 1 μM, 10 μM, 100 μM, 500 μM and 1 mM. The OD600 of each sample was measured before harvesting the full 1-ml culture at 9 h for analysis.

Both EMAL283 and EMAL329 show optimal YFP or FdeR production at intermediate concentrations of IPTG (500 μM and 100 μM, respectively), with higher concentrations leading to reduced protein levels (FIG. 12C). By contrast, EMAL284 and EMAL336, containing OptoLAC1B and OptoLAC2B, respectively, show maximum production in full darkness with a monotonic light response, which facilitates tunability (FIG. 12C).

Note, in the above examples, for Sodium dodecyl sulfate polyacrylamide gel electrophoresis, western blotting and protein quantification, samples were prepared by centrifuging 1-ml cell cultures at 17,000 r.c.f. for 5 min in a benchtop microcentrifuge (Thermo Scientific, Sorvall Legend Micro 17) at room temperature. The pellets were resuspended in 200 μl of resuspension buffer (Tris 50 mM, pH 8.0 and 300 mM NaCl), mixed with 50 μl of SDS sample buffer, and incubated at 100° C. in a heat block (Eppendorf ThermoMixer C) for 10 min at 700 r.p.m. Samples were loaded onto 12% SDS-PAGE gels and resolved. The volumes loaded on the gels (between 3 and 20 μl) were adjusted based on the OD600 measured just before the samples were harvested (between 0.5-3) to load the same amount of total cell mass, equivalent to 10 μl of sample from cells harvested at a final OD600=1.

Following electrophoresis, gels were either stained with Coomassie Brilliant Blue G-250 (Thermo Fisher), used as loading controls or analyzed by western blot following previously described methods54. Proteins were transferred onto PVDF membranes using a Trans-Blot Turbo Transfer System (Bio-Rad) using the standard protocol. LacI protein expression was assayed using a mouse anti-LacI antibody (Abcam, ab33832; 1/4,000) and goat anti-mouse secondary antibody (Abcam, ab205719; 1/10,000). YFP and FdeR expression were assayed using a mouse anti-His tag antibody (GenScript, A00612; 1/10,000). Blots were revealed using Clarity ECL substrate (Bio-Rad) according to the manufacturer's instructions. Images were taken using a Bio-Rad ChemiDoc MP Imaging System with Image Lab software, using the chemiluminescence protocol. Pixel intensity was quantified using ImageJ software. For all western blots, identically loaded gels were run and stained with Coomassie Brilliant Blue to confirm equal protein loading.

OptoLAC1B and OptoLAC2B can be induced with IPTG even when cells are kept in blue light, but this significantly hampers cell growth, probably due to the burden imposed by the strong T7 polymerase when inhibiting an already destabilized LacI.

### Other Examples of Recombinant Protein Production Using OptoLAC Circuits

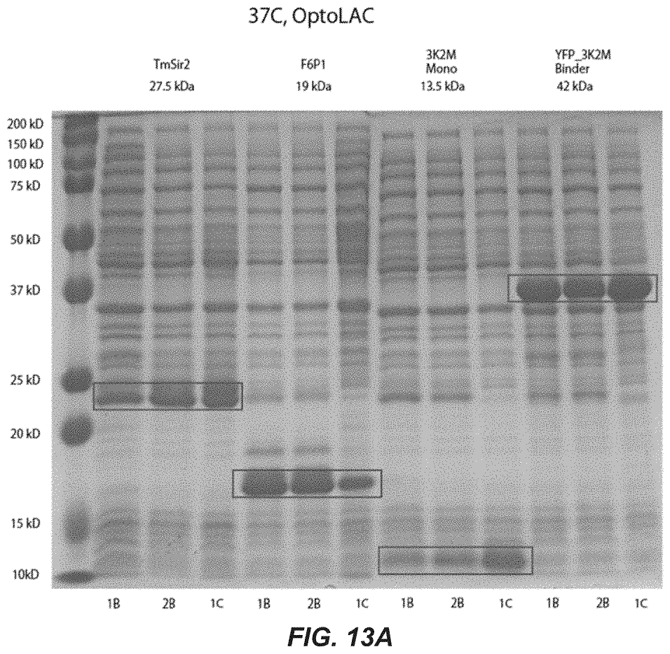

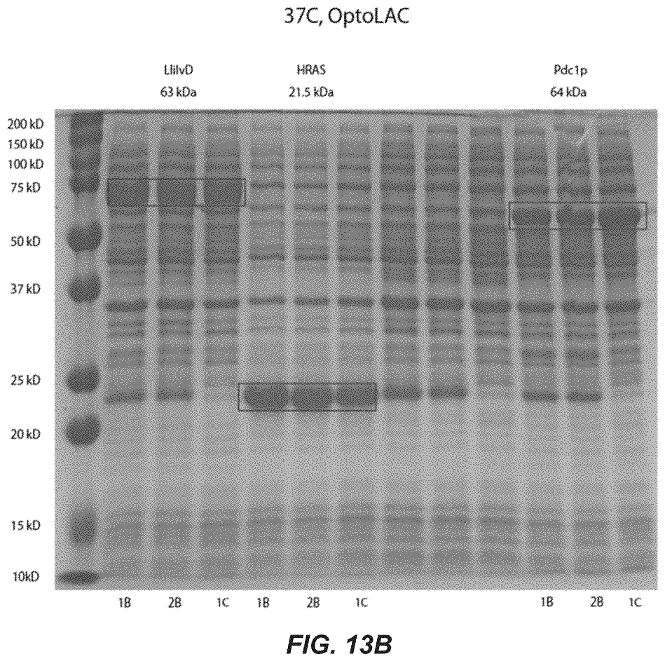

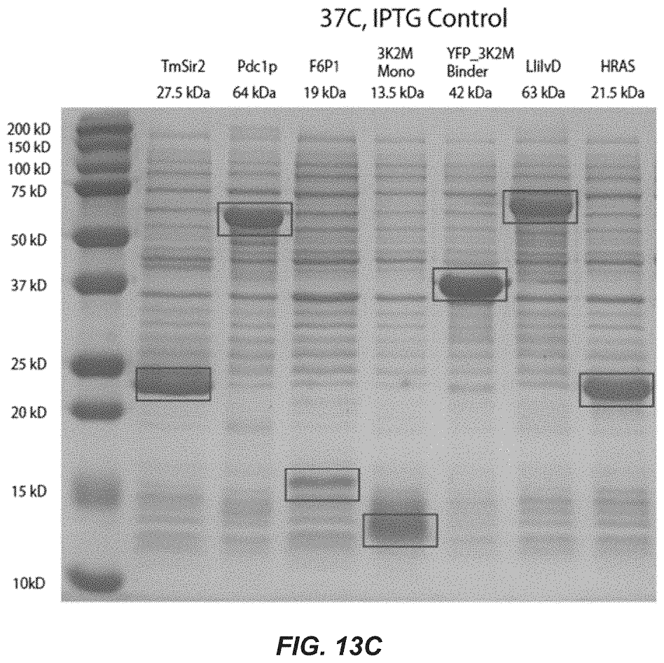

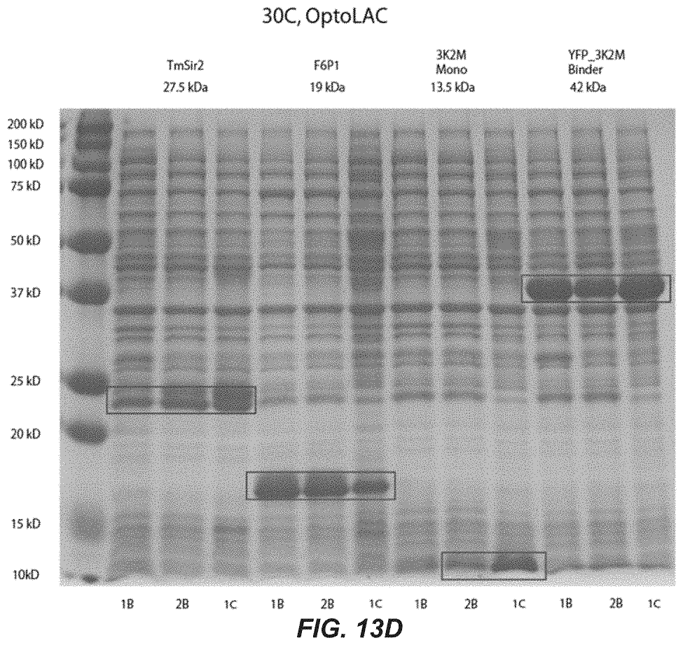

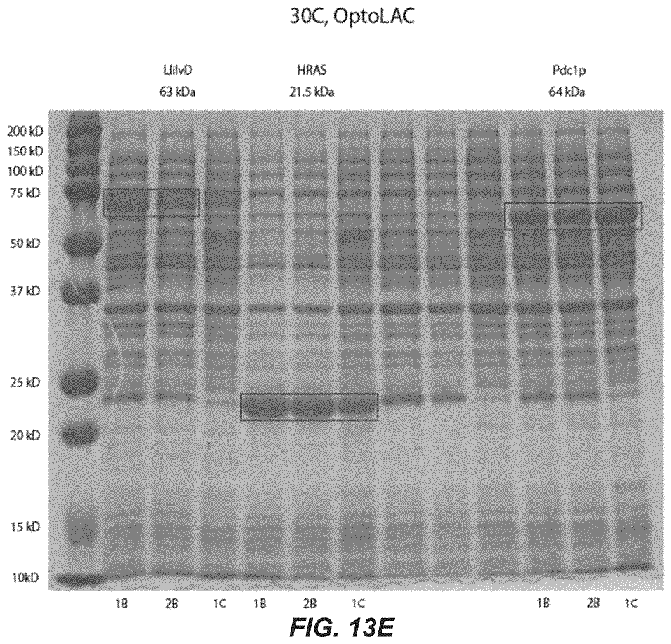

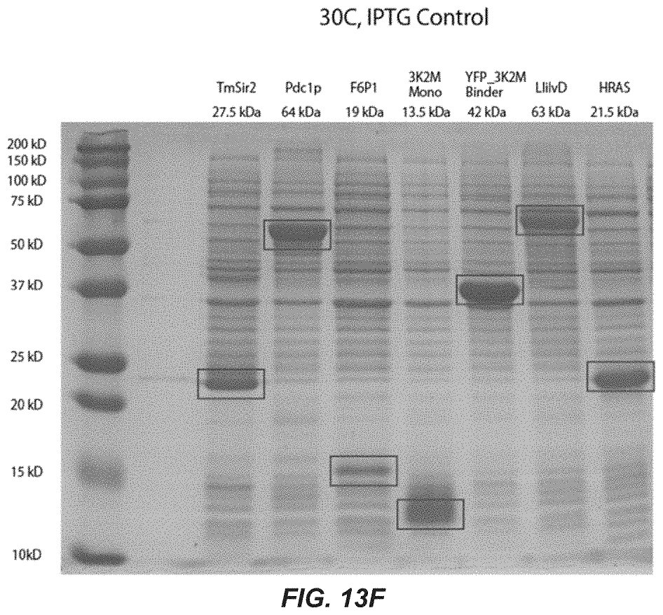

Referring to FIGS. 13A-13F, a compilation of gels can be seen showing the production of various proteins. Cultures were grown under continuous blue light until exponential phase, then moved to darkness (wrapped in aluminum foil) and grown for 8 hours at 37° C. (FIGS. 13A-13C) or 20 hours at 30° C. (FIGS. 13D-13F). Controls were grown in ambient light and induced in exponential phase by the addition of IPTG to a final concentration of 1 mM. The various circuits include OptoLAC1B=SsrA-LAA degradation tag, p15A origin of replication; OptoLAC2B=SsrA-AAV degradation tag, p15A origin of replication; OptoLAC1C=SsrA-LAA degradation tag, pSC101 origin of replication; TmSir2=sirtuin from Thermotoga maritima; F6P1=Pdc1p-binding nanobody; 3K2M Mono=SH2 domain-binding monobody; YFP_3K2M Binder=YFP−SH2 fusion; LlilvD=dihydroxy-acid dehydratase from Lactococcus lactis; HRAS=GTPase HRas; and Pdc1p=pyruvate decarboxylase from Saccharomyces cerevisiae.

Optogenetics may help balance the levels of expression of different metabolic enzymes, which greatly impacts the performance of engineered pathways. This key challenge in metabolic engineering is currently addressed by assembling large numbers of constructs, usually in biofoundries, to combinatorially test different enzyme expression levels. However, the ability of OptoLAC circuits to finely tune gene expression from different promoters could transform this practice by testing varying pulses of light instead of copious numbers of constitutive constructs.

The testable expression space of the disclosed OptoLAC circuits could be further expanded using mutations that sensitize or invert the light response of the YF1 protein.

One OptoLAC circuit can be combined with other circuits that respond orthogonally to different wavelengths of light (for example, green, red or infrared), to enable multivariate modular metabolic engineering to balance pathways. Such a polychromatic approach would not only dramatically reduce the number of constructs required to balance complex metabolic pathways, but also increase the resolution at which levels of enzyme expression could be screened for pathway optimization.

Light penetration is unlikely to be as limiting to the disclosed systems as it is for photosynthetic organisms. In cell cultures of algae or cyanobacteria, there is a stoichiometric relationship between the photons absorbed by the photosynthetic systems and adenosine triphosphate output. The light-harvesting antennas in photosynthetic organisms present an additional challenge to effectively illuminate cultures for robust cell growth and production. In contrast, the disclosed optogenetic circuits are not required for energy metabolism and E. coli has not evolved to compete for light harvesting. Instead, the disclosed circuits control expression of a first repressor (such as cI expression), which is hypothesized as leading to accumulation of LacI mRNA and protein under blue light that does not immediately subside when cells are switched to darkness. In this sense, the disclosed optogenetic circuits have an inherent ‘memory’ of having been exposed to blue light, which makes the requirements for light penetration more lenient. This memory is evident in kinetics experiments (FIG. 8D), bioreactor fermentations (FIG. 11C) and protein production (FIGS. 12A, 12B). In all these instances, the response of the disclosed circuits has an ˜2-h delay, as opposed to the almost immediate response to IPTG as an allosteric Lad inhibitor.

In addition to alleviating demanding light penetration conditions, this feature of OptoLAC circuits may also be useful to mimic slow-acting inducible systems, such as those based on quorum sensing, which have been effectively used as auto-inducible systems for chemical production. In some embodiments, a gradual decrease of illumination could be used to mirror the gradual increase of quorum-sensing molecule concentrations, but with the advantage that the onset and rate of light dimming can be easily controlled and operated reversibly using the disclosed OptoLAC circuits.

In some embodiments, autoinduction strategies may also be possible in which genes are gradually expressed as increasing optical densities reduce light penetration in large bioreactors.

In addition to being effective for metabolic engineering in K-12 strains, OptoLAC circuits are also applicable for recombinant protein production in B-strains. Light controls may be particularly useful for recombinant proteins that are difficult to produce. For example, proteins toxic to E. coli are often produced at very low yields or require the addition of inhibitors against their toxic activity. Additionally, rates of protein expression can be too high to keep recombinant proteins from aggregating or forming inclusion bodies. In some embodiments, this problem can sometimes be alleviated by inducing at low temperatures and/or using autoinduction media. However, the fine tunability of gene expression afforded by light could potentially enhance the yields of toxic proteins by tempering their expression levels, or improve the quality and solubility of proteins that are difficult to produce by better regulating their rates of expression.

OptoLAC circuits open the door to replacing IPTG with light for a broad number of applications. In industrial fermentations, eliminating IPTG (or other chemical inducers) would reduce costs. Additionally, it would enable dynamic controls in processes for which the use of chemical inducers is prohibitively expensive.

Beyond chemical and recombinant protein production, IPTG induction has been used for a variety of research applications, including persistence, bacterial motility, inducible protein degradation, and biofilm formation. Furthermore, the LacI repressor has been exported to other bacteria for IPTG inducibility. Therefore, OptoLAC circuits may be applicable in a wide range of systems originally designed to be IPTG-inducible, providing them with the enhanced capabilities typically afforded by light, such as fine tunability, reversibility and spatiotemporal control.

Those skilled in the art will recognize, or be able to ascertain using no more than routine experimentation, many equivalents to the specific embodiments of the invention described herein. Such equivalents are intended to be encompassed by the following claims.

What is claimed is:

1. A system for controlling expression of a gene of interest, comprising:
a first sequence encoding a first repressor, under a first promoter, the first promoter being light-controllable promoter;
a second sequence encoding a second repressor, under a second promoter, the second promoter being controllable by the first repressor; and
a third sequence encoding a gene of interest under a third promoter, the third promoter being controllable by the second repressor.

2. The system according to claim 1, wherein the first promoter is controlled by a two-component system or a one-component system.

3. The system according to claim 2, wherein the two-component system comprises the transcription factor FixJ, which in turn is controlled by the light responsive kinase/phosphatase YF1; or wherein the one-component system comprises the LOV-domain-containing EL222 transcription factor fused to an activation domain.

4. The system according to claim 1, wherein the first repressor is phage repressor cI, the second repressor is a LacI repressor, or a combination thereof.

5. The system according to claim 1, wherein the third promoter is a lacO-operator-containing promoter.

6. The system according to claim 1, wherein the first sequence further encodes a degradation tag fused to the first repressor, the second sequence further encodes a degradation tag fused to the second repressor, or a combination thereof.

7. The system according to claim 1, wherein the first promoter is also controllable by the LacI repressor, comprises PFD_{FixK2}, or a combination thereof.

8. The system according to claim 1, wherein the gene of interest is one or more genes involved in the biosynthesis of a chemical compound.

9. The system according to claim 8, wherein the chemical compound is mevalonate or isobutanol.

10. The system according to claim 1, wherein the gene of interest is one or more genes involved in production of a recombinant protein.

11. The system according to claim 10, wherein the recombinant protein comprises a fluorescent protein, FdeR, TmSir2, Pdc1p, nanobodies, monobodies, LlilvD, HRAS, or a combination thereof.

12. The system according to claim 1, wherein the system is present in at least one plasmid, each plasmid being free of endogenous and constitutive sequences encoding the first or second repressors, and each plasmid independently including the first sequence, second sequence, third sequence, or a combination thereof.

13. The system according to claim 1, wherein the system is present in or integrated into the genome of an engineered microorganism, the engineered microorganism being free of endogenous and constitutive sequences encoding the first or second repressors.

14. The system according to claim 13, wherein the engineered microorganism is a strain of E. coli.

15. The system according to claim 1, wherein a portion of the system is integrated into the genome of an engineered microorganism, the engineered microorganism being free of endogenous and constitutive sequences encoding the first or second repressors; and wherein the remaining portion of the system is present in at least one plasmid, each plasmid being free of endogenous and constitutive sequences encoding the first or second repressors, and each plasmid including the first sequence, second sequence, third sequence, or a combination thereof.

16. A method for controlling chemical or protein production, comprising the steps of:
growing engineered microorganism cells under a first lighting condition, the engineered microorganism cells containing:
a first sequence encoding a first repressor, under a first promoter, the first promoter being light-controllable;
a second sequence encoding a second repressor, under a second promoter, the second promoter being controllable by the first repressor; and
a third sequence encoding a gene of interest under a third promoter, the third promoter being controllable by the second repressor,
the engineered microorganism cells being free of endogenous and constitutive sequences encoding the first or second repressors;
expressing the gene of interest by adjusting the first lighting condition and causing the first repressor to be expressed, preventing the second repressor from being expressed, allowing the third promoter to be activated.

17. The method according to claim 16, wherein the first lighting condition is adjusted when the optical density at a wavelength of 600 nm (OD₆₀₀) of the microorganism is determined to be within a predetermined range.

18. The method according to claim 17, wherein the predetermined range of OD₆₀₀ is between 0.1 and 2.

19. The method according to claim 16, further comprising obtaining cell-free supernatant containing a chemical of interest expressed by the gene of interest.

20. The method according to claim 16, further comprising collecting the cells and lysing the cells to obtain a chemical or protein of interest produced by the gene of interest.

21. The method according to claim 16, further comprising repeatedly turning on a light source for a first period of time T₁, then turning off a light source for a second period of time T₂.

22. The method according to claim 21, wherein T₁/(T₁+T₂) is between about 0.001 and about 0.1.

23. A kit, comprising:
one or more strains of engineered microorganisms, each having a first nucleotide sequence integrated; the first nucleotide sequence encoding a first repressor, under a first promoter, the first promoter being light-controllable promoter; and a second nucleotide sequence encoding a second repressor under a second promoter, the second promoter being controllable by the first repressor, and
one or more plasmids, each plasmid containing a different promoter controlled by the second repressor, different multicloning sites to integrate one or more genes of interest, at least one optional sequence to fuse tags, and a different origin of replication, where each plasmid is free of endogenous and constitutive sequences encoding the first or second repressors.


SEQUENCE LISTING (4 sequences)

SEQ ID NO: 1 - DNA, 282 bp, Artificial Sequence
  Description of Artificial Sequence Synthetic polynucleotide
  Promoter P_FixK2_lacO
acgcccgtgatcctgatcaccggctatccggacgaaaacatctcgacccgggccgccgaggccggcgtaaaagacgtggttttgaagccgcttctcgacgaaaacctgctcaagcgtatccgccgcgccatccaggaccggcctcgggcatgacctacggggttctacgtaaggcaccccccttaagatatcgctcgaaattttcgaacctcccgataccgcgtaccaatgcgtcatcacaacggagttgtgagcggataacaaactagtaaagaggagaaa

SEQ ID NO: 2 - DNA, 4666 bp, Artificial Sequence
  Description of Artificial Sequence Synthetic polynucleotide
  OptoLAC1
tcaatcgttgagcatgccggcgcgcatcgcgaggcgaaccagctccgaaaggctgttggcctgcatcttggtcatgacgttggcccgatacacctcgatggtgcgcgggctgatgtcgtactcgcgggcgatcagcttgttggaaaggccggcgatcagcccttccatgacctggcgctccctggggctcaacgaggcgacgcgggcggcgatatcctgcgcgacggcctcgctcttggcggccggctcggcctggcggatcgccgattcgatcatggcggtgaggcggtcgtcctcgaaaggcttttccagaaagtccaccgcccctaacttcatcgcctcgaccgcgagcggcacgtcgccgtgaccggtcatgatgaggatcggaaaggggctttgctgcgccttcatccgcttcaacagctcgatgccgtcaaggcccggcatgcgcacgtcggagacgacgcagccgaaggagagacccggcagggcgtcgagaaaggcttgcgcgtcgtcaaacagcgtgacgccgaagccggcagaatccagcaggaaattcagcgaatcccgcatcgccgcgtcgtcgtcgatgacgtagatatgtcccttggtcgtcatgctagacctcctatcatctcgtcggctgccgggagggtgaagcggaaggtcgccccgcccgatgcgttgctctcggcccacatgcgcccgccgtgagcttcgatgatcgagcggctgatggacagtcccacgcccatgccggtgtccttggtggtgaagaaagtctgaaacaggttcggaatgacgtcgtcctggaaaccgctgccggtgtcggacacttcgacctcgatcatgtcgtcggcggcgggggtgttggtgacgacgagctcgcgtcgctgcgactgagccatcgcttccagcgcgttgcggaacaggttgaccaggacctgctggatctgcacccggtcggcgagaacgagatcggcgcccggatcgagactgaagcggagctgcacgttctgctcgcgcgcgccggcaagcccgagcgcgccggcctcctcgatcagcttggagagactctcgacccgcttctccgattcgccgcgggcaacgaagtcgcgcaggcgccggatgatctggccggcgcgcagcgcctgctcggcggcgcggtccagggcgctttcgaccttcggtgtgttcggatcactgctgccggcaagcagccgccgcgagcccttcatgtagttgctgatcgccgccagcggctggttgagctcgtgcgcgagcgcggacgccatttcgcccatggcgctcagcctggagacgtggacgagctcggattgcagttcctggaggcgcgcctgggtctgctggtgctcggtgatatcattctgaataccgacaaaatacgttttatcctctatttccattggatcaatatttaattcattccagaacatcgttccgtcttttttgtagttttggatctgaacggtgaccggttctttattttgtaaagcggttctgatgttgtccacttctgcaggatctgtgtgtttcccctgtaagaagcgacagttctttcctaaaatttcctcggtctcgtagccggtcatttgaacaaagccttgatttacgtagacaataggattatcttcaagtgcgggatctgtaattaccacaccgactcgcacgtgatcaagtgcttttttgatgacttccagctgtcctggtatcccaaatgattgaaaactagccacattcaccaccctcaattgactctcttccgggcgctatcatgccataccgcgaaaggttttgcaccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctacgcccgtgatcctgatcaccggctatccggacgaaaacatctcgacccgggccgccgaggccggcgtaaaagacgtggttttgaagccgcttctcgacgaaaacctgctcaagcgtatccgccgcgccatccaggaccggcctcgggcatgacctacggggttctacgtaaggcaccccccttaagatatcgctcgaaattttcgaacctcccgataccgcgtaccaatgcgtcatcacaacggagtactagtaaagaggagaaatactagatgagcacaaaaaagaaaccattaacacaagagcagcttgaggacgcacgtcgccttaaagcaatttatgaaaaaaagaaaaatgaacttggcttatcccaggaatctgtcgcagacaagatggggatggggcagtcaggcgttggtgctttatttaatggcatcaatgcattaaatgcttataacgccgcattgcttgcaaaaattctcaaagttagcgttgaagaatttagcccttcaatcgccagagaaatctacgagatgtatgaagcggttagtatgcagccgtcacttagaagtgagtatgagtaccctgttttttctcatgttcaggcagggatgttctcacctgagcttagaacctttaccaaaggtgatgcggagagatgggtaagcacaaccaaaaaagccagtgattctgcattctggcttgaggttgaaggtaattccatgaccgcaccaacaggctccaagccgagctttcctgacggaatgttaattctcgttgaccctgagcaggctgttgagccaggtgatttctgcatagccagacttgggggtgatgagtttaccttcaagaaactgatcagggatagcggtcaggtgtttttacaaccactaaacccacagtacccaatgatcccatgcaatgagagttgttccgttgtggggaaagttatcgctagtcagtggcctgaagagacgtttggcgctgcaaacgacgaaaactacgctttagtagcttaataataacgctgatagtgctagtgtagatcgctactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagtaacaccgtgcgtgttgactattttacctctggcggtgataatggttgctctagaaataattttgtttaactttaagaaggagagctagcatgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcaggcagcaaacgacgaaaactacgctgcagcagtttaatgagcaccaccaccaccaccactgagatccggctgctaacaaagcccgaaaggaagctgagttggctgctgccaccgctgagcaataactagcataaccccttggggcctctaaacgggtcttgaggggttttttg

SEQ ID NO: 3 - DNA, 4663 bp, Artificial Sequence
  Description of Artificial Sequence Synthetic polynucleotide
  OptoLAC2
tcaatcgttgagcatgccggcgcgcatcgcgaggcgaaccagctccgaaaggctgttggcctgcatcttggtcatgacgttggcccgatacacctcgatggtgcgcgggctgatgtcgtactcgcgggcgatcagcttgttggaaaggccggcgatcagcccttccatgacctggcgctccctggggctcaacgaggcgacgcgggcggcgatatcctgcgcgacggcctcgctcttggcggccggctcggcctggcggatcgccgattcgatcatggcggtgaggcggtcgtcctcgaaaggcttttccagaaagtccaccgcccctaacttcatcgcctcgaccgcgagcggcacgtcgccgtgaccggtcatgatgaggatcggaaaggggctttgctgcgccttcatccgcttcaacagctcgatgccgtcaaggcccggcatgcgcacgtcggagacgacgcagccgaaggagagacccggcagggcgtcgagaaaggcttgcgcgtcgtcaaacagcgtgacgccgaagccggcagaatccagcaggaaattcagcgaatcccgcatcgccgcgtcgtcgtcgatgacgtagatatgtcccttggtcgtcatgctagacctcctatcatctcgtcggctgccgggagggtgaagcggaaggtcgccccgcccgatgcgttgctctcggcccacatgcgcccgccgtgagcttcgatgatcgagcggctgatggacagtcccacgcccatgccggtgtccttggtggtgaagaaagtctgaaacaggttcggaatgacgtcgtcctggaaaccgctgccggtgtcggacacttcgacctcgatcatgtcgtcggcggcgggggtgttggtgacgacgagctcgcgtcgctgcgactgagccatcgcttccagcgcgttgcggaacaggttgaccaggacctgctggatctgcacccggtcggcgagaacgagatcggcgcccggatcgagactgaagcggagctgcacgttctgctcgcgcgcgccggcaagcccgagcgcgccggcctcctcgatcagcttggagagactctcgacccgcttctccgattcgccgcgggcaacgaagtcgcgcaggcgccggatgatctggccggcgcgcagcgcctgctcggcggcgcggtccagggcgctttcgaccttcggtgtgttcggatcactgctgccggcaagcagccgccgcgagcccttcatgtagttgctgatcgccgccagcggctggttgagctcgtgcgcgagcgcggacgccatttcgcccatggcgctcagcctggagacgtggacgagctcggattgcagttcctggaggcgcgcctgggtctgctggtgctcggtgatatcattctgaataccgacaaaatacgttttatcctctatttccattggatcaatatttaattcattccagaacatcgttccgtcttttttgtagttttggatctgaacggtgaccggttctttattttgtaaagcggttctgatgttgtccacttctgcaggatctgtgtgtttcccctgtaagaagcgacagttctttcctaaaatttcctcggtctcgtagccggtcatttgaacaaagccttgatttacgtagacaataggattatcttcaagtgcgggatctgtaattaccacaccgactcgcacgtgatcaagtgcttttttgatgacttccagctgtcctggtatcccaaatgattgaaaactagccacattcaccaccctcaattgactctcttccgggcgctatcatgccataccgcgaaaggttttgcaccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatctacgcccgtgatcctgatcaccggctatccggacgaaaacatctcgacccgggccgccgaggccggcgtaaaagacgtggttttgaagccgcttctcgacgaaaacctgctcaagcgtatccgccgcgccatccaggaccggcctcgggcatgacctacggggttctacgtaaggcaccccccttaagatatcgctcgaaattttcgaacctcccgataccgcgtaccaatgcgtcatcacaacggagtactagtaaagaggagaaatactagatgagcacaaaaaagaaaccattaacacaagagcagcttgaggacgcacgtcgccttaaagcaatttatgaaaaaaagaaaaatgaacttggcttatcccaggaatctgtcgcagacaagatggggatggggcagtcaggcgttggtgctttatttaatggcatcaatgcattaaatgcttataacgccgcattgcttgcaaaaattctcaaagttagcgttgaagaatttagcccttcaatcgccagagaaatctacgagatgtatgaagcggttagtatgcagccgtcacttagaagtgagtatgagtaccctgttttttctcatgttcaggcagggatgttctcacctgagcttagaacctttaccaaaggtgatgcggagagatgggtaagcacaaccaaaaaagccagtgattctgcattctggcttgaggttgaaggtaattccatgaccgcaccaacaggctccaagccgagctttcctgacggaatgttaattctcgttgaccctgagcaggctgttgagccaggtgatttctgcatagccagacttgggggtgatgagtttaccttcaagaaactgatcagggatagcggtcaggtgtttttacaaccactaaacccacagtacccaatgatcccatgcaatgagagttgttccgttgtggggaaagttatcgctagtcagtggcctgaagagacgtttggcgctgcaaacgacgaaaactacgctttagtagcttaataacgctgatagtgctagtgtagatcgctactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagtaacaccgtgcgtgttgactattttacctctggcggtgataatggttgctctagaaataattttgtttaactttaagaaggagagctagcatgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcaggcagcaaacgacgaaaactacgctgcagcagtttaatgagcaccaccaccaccaccactgagatccggctgctaacaaagcccgaaaggaagctgagttggctgctgccaccgctgagcaataactagcataaccccttggggcctctaaacgggtcttgaggggttttttg

SEQ ID NO: 4 - DNA, 4496 bp, Artificial Sequence
  Description of Artificial Sequence Synthetic polynucleotide
  OptoLAC3
gacaccatcgaatggtgcaaaacctttcgcggtatggcatgatagcgcccggaagagagtcaattgagggtggtgaatgtggctagttttcaatcatttgggataccaggacagctggaagtcatcaaaaaagcacttgatcacgtgcgagtcggtgtggtaattacagatcccgcacttgaagataatcctattgtctacgtaaatcaaggctttgttcaaatgaccggctacgagaccgaggaaattttaggaaagaactgtcgcttcttacaggggaaacacacagatcctgcagaagtggacaacatcagaaccgctttacaaaataaagaaccggtcaccgttcagatccaaaactacaaaaaagacggaacgatgttctggaatgaattaaatattgatccaatggaaatagaggataaaacgtattttgtcggaattcagaatgatatcaccgagcaccagcagacccaggcgcgcctccaggaactgcaatccgagctcgtccacgtctccaggctgagcgccatgggcgaaatggcgtccgcgctcgcgcacgagctcaaccagccgctggcggcgatcagcaactacatgaagggctcgcggcggctgcttgccggcagcagtgatccgaacacaccgaaggtcgaaagcgccctggaccgcgccgccgagcaggcgctgcgcgccggccagatcatccggcgcctgcgcgacttcgttgcccgcggcgaatcggagaagcgggtcgagagtctctccaagctgatcgaggaggccggcgcgctcgggcttgccggcgcgcgcgagcagaacgtgcagctccgcttcagtctcgatccgggcgccgatctcgttctcgccgaccgggtgcagatccagcaggtcctggtcaacctgttccgcaacgcgctggaagcgatggctcagtcgcagcgacgcgagctcgtcgtcaccaacacccccgccgccgacgacatgatcgaggtcgaagtgtccgacaccggcagcggtttccaggacgacgtcattccgaacctgtttcagactttcttcaccaccaaggacaccggcatgggcgtgggactgtccatcagccgctcgatcatcgaagctcacggcgggcgcatgtgggccgagagcaacgcatcgggcggggcgaccttccgcttcaccctcccggcagccgacgagatgataggaggtctagcatgacgaccaagggacatatctacgtcatcgacgacgacgcggcgatgcgggattcgctgaatttcctgctggattctgccggcttcggcgtcacgctgtttgacgacgcgcaagcctttctcgacgccctgccgggtctctccttcggctgcgtcgtctccgacgtgcgcatgccgggccttgacggcatcgagctgttgaagcggatgaaggcgcagcaaagcccctttccgatcctcatcatgaccggtcacggcgacgtgccgctcgcggtcgaggcgatgaagttaggggcggtggactttctggaaaagcctttcgaggacgaccgcctcaccgccatgatcgaatcggcgatccgccaggccgagccggccgccaagagcgaggccgtcgcgcaggatatcgccgcccgcgtcgcctcgttgagccccagggagcgccaggtcatggaagggctgatcgccggcctttccaacaagctgatcgcccgcgagtacgacatcagcccgcgcaccatcgaggtgtatcgggccaacgtcatgaccaagatgcaggccaacagcctttcggagctggttcgcctcgcgatgcgcgccggcatgctcaacgattgacaattgatgtaagttagctcactcattaggcaccgggatctcgaccgatgcccttgagagccttcaacccagtctgtacgcggccgcgtccggcgtagaggatcgagatctacgcccgtgatcctgatcaccggctatccggacgaaaacatctcgacccgggccgccgaggccggcgtaaaagacgtggttttgaagccgcttctcgacgaaaacctgctcaagcgtatccgccgcgccatccaggaccggcctcgggcatgacctacggggttctacgtaaggcaccccccttaagatatcgctcgaaattttcgaacctcccgataccgcgtaccaatgcgtcatcacaacggagttgtgagcggataacaaactagtaaagaggagaaatactagatgagcacaaaaaagaaaccattaacacaagagcagcttgaggacgcacgtcgccttaaagcaatttatgaaaaaaagaaaaatgaacttggcttatcccaggaatctgtcgcagacaagatggggatggggcagtcaggcgttggtgctttatttaatggcatcaatgcattaaatgcttataacgccgcattgcttgcaaaaattctcaaagttagcgttgaagaatttagcccttcaatcgccagagaaatctacgagatgtatgaagcggttagtatgcagccgtcacttagaagtgagtatgagtaccctgttttttctcatgttcaggcagggatgttctcacctgagcttagaacctttaccaaaggtgatgcggagagatgggtaagcacaaccaaaaaagccagtgattctgcattctggcttgaggttgaaggtaattccatgaccgcaccaacaggctccaagccgagctttcctgacggaatgttaattctcgttgaccctgagcaggctgttgagccaggtgatttctgcatagccagacttgggggtgatgagtttaccttcaagaaactgatcagggatagcggtcaggtgtttttacaaccactaaacccacagtacccaatgatcccatgcaatgagagttgttccgttgtggggaaagttatcgctagtcagtggcctgaagagacgtttggcgctgcaaacgacgaaaactacgctttagtagcttaataacgctgatagtgctagtgtagatcgctactagagccaggcatcaaataaaacgaaaggctcagtcgaaagactgggcctttcgttttatctgttgtttgtcggtgaacgctctctactagagtcacactggctcaccttcgggtgggcctttctgcgtttatatactagagtaacaccgtgcgtgttgactattttacctctggcggtgataatggttgctctagaaataattttgtttaactttaagaaggagagctagcatgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcaggcagcaaacgacgaaaactacgctgcagcagtttaatgagcaccaccaccaccaccactgagatccggctgctaacaaagcccgaaaggaagctgagttggctgctgccaccgctgagcaataactagcataaccccttggggcctctaaacgggtcttgaggggttttttg